(19) United States
(12) Patent Application Publication
(10) Pub. No.: US 2021/0327562 A1
(43) Pub. Date: Oct. 21, 2021

(54) ARTIFICIAL INTELLIGENCE DRIVEN RAPID TESTING SYSTEM FOR INFECTIOUS DISEASES

(71) Applicant: PREDICTMEDIX INC., Toronto (CA)

(72) Inventors: Rahul Kushwah, Toronto (CA); Sheldon Kales, Toronto (CA); Nandan Mishra, Noida (IN); Himanshu Ujjawal Singh, Malviya Nagar (IN)

(21) Appl. No.: 17/235,471

(22) Filed: Apr. 20, 2021

Related U.S. Application Data
(60) Provisional application No. 63/012,510, filed on Apr. 20, 2020.
(60) Provisional application No. 63/048,131, filed on Jul. 5, 2020.
(60) Provisional application No. 63/048,152, filed on Jul. 5, 2020.
(60) Provisional application No. 63/058,567, filed on Jul. 30, 2020.
(60) Provisional application No. 63/072,392, filed on Aug. 31, 2020.

(51) Int. Cl. G16H 30/40; G16H 30/20; G16H 50/80; G16H 50/20; G16H 10/60; G06K 9/62; G06T 7/00

(52) U.S. Cl. CPC G16H 30/40; G16H 30/20; G16H 50/80; G16H 50/20; G06T 2207/10048; G06K 9/6256; G06T 7/0012; G06T 2207/10081; G16H 10/60

(57) ABSTRACT

A method and system for rapid scanning, surveillance and detection of an infectious disease in an individual is disclosed. The method and system operate a processor to receive a set of training data comprising training data to train one or more analytical models using artificial intelligence algorithms. The method and system then use one or more input devices to receive parameters related to the individual such as images, body temperature, clinical data, CT scan data, voice data, video data and thermal image. Each parameter is associated to one or more features. An analytical model is selected from one or more analytical models to detect the presence of the infectious disease in an individual. The method and system for rapid scanning, surveillance and detection can be trained on a wide dataset received from clinical data, CT-scan, X-ray, temperature sensor and other data.

### RELATED APPLICATIONS

This application is related to the following:

1. Provisional Application Ser. No. 63/012,510, filed Apr. 20, 2020 (Parent Provisional);

2. Provisional Application Ser. No. 63/048,131, filed July 5, 2020;

3. Provisional Application Ser. No. 63/048,152, filed Jul. 5, 2020;

4. Provisional Application Ser. No. 63/058,567, filed Jul. 30, 2020;

5. Provisional Application Ser. No. 63/072,392, filed Aug. 31, 2020;

This application claims priority to the Parent Provisional, and hereby claims benefit of the filing date thereof pursuant to 37 CFR § 1.78(a)(4).

The subject matter of the Parent Provisional and the Related Application, each in its entirety, is expressly incorporated herein.

### BACKGROUND OF THE INVENTION

Identification and tracking of infectious diseases during an outbreak of a global pandemic is an important method of controlling the spread of disease across geographical areas. Infectious diseases, for example, influenza, COVID-19 and other viral infections, which are highly infectious are resulting in human catastrophes and innumerable loss of human lives. Further, there are very few drugs for treatment of such viral infections. Therefore, such infectious diseases need to be identified, controlled and monitored through timely surveillance. Certain Infectious disease such as COVID-19 for which there is still no cure, medicines or vaccines can pose a serious threat to a community, locality, city, region or a country.

During spread of such a disease or pandemic, an effective response strategy depends upon identification of infected cases and isolation of these infected cases to break the chain of spread of the disease. Additionally, it is equally important to monitor the recovery of the infected cases. An effective surveillance to stop the spread of the infectious disease either through mass testing or using technology such as thermal scanner or AI enabled scanning systems provide an effective means to control the spread of disease. Furthermore, the identification, tracing and tracking of direct contact of the infected cases and quarantining these direct contacts are important steps to control the spread of the infectious disease.

An infectious disease can spread by coughing, sneezing or dispersal of droplets released by an infected individual or a patient. Currently, there is no specific treatment method, drug, or proven vaccine for COVID-19. Current vaccines have been allowed under emergency use and would be in use until an effective approved vaccine is developed. Therefore, containment of the infectious disease is the most effective solution. It is the only way currently to control the disease. The only available method is to identify individuals having or carrying infection through testing and then isolating them so that the infection is not spread within the community.

The level of infection in an individual depends upon the immune system of an individual. Some individuals may be partially affected by the infectious disease such as COVID-19 and show little symptoms, while other set of people may show severe symptoms of the infectious disease. Another set of people may not be affected but may act as carrier of the disease. Therefore, in the interest of society, it is desirable to identify individuals that show symptoms of the disease or are carriers of the disease. Identification of asymptomatic individuals in places where there is large congregation of people such as hospitals, airports, transit terminals, office buildings, distribution centers, stadiums, etc. is key to containing disease spread. Therefore, a rapid scanning system is needed that can identify, track, and isolate the infected individuals from a large group at a rapid pace.

Identification, isolating, quarantining and monitoring these infection cases requires a good and effective surveillance system with a well-defined protocol and management. One of the surveillance methods and/or systems could be implemented by analyzing the physical parameters such as temperature, voice, cough and body scanning in a contactless manner. In addition, if the preliminary results through physical examination are positive, further medical tests may be performed to monitor the health of the individual.

One way is to use technology to scan a large group of people at a very fast rate, for example, by using a thermal scanning which is an effective way of scanning. However, this is not fool proof as an individual can easily bypass the simple system by taking fever reducing over the counter drugs.

In addition, monitoring the condition of each individual/patient until recovery and providing effective care is important to stop the spread of the infectious disease and to control the death rate. To achieve this objective, a large scale surveillance system is required that can be used for community surveillance, locality surveillance or a regional surveillance that can provide information and intelligence to formulate an effective response strategy, for example, isolation of hotspots, breaking the chain of infection by containment zones, and planning for human movements.

Another way is to use multiple scanning devices coupled with artificial intelligence to identify the likelihood of the person being infected by an infectious disease. Artificial intelligence coupled to a good surveillance method and/or system that provides for large scale scanning and. prediction of the likelihood of infectious diseases in an individual can prevent an outbreak, control the spread of infectious disease and allow effective monitoring of patients. In addition, the collected patient data may be useful and important for management and containment of the infectious disease.

### Problem Statement

A need for a fast, scalable and efficient method and system is required for large scale scanning of individuals in a contactless manner that can be linked with medical infrastructure and can facilitate the efficient and effective monitoring of the infectious disease.

### Solution

The present invention provides a novel method and system of infectious disease surveillance. The method and system utilize physical scanning of an individual to assess the likelihood that the individual is suffering from an infectious disease. The method and system of infectious disease surveillance can be deployed at airports, railway stations, malls, bus stands, cinema halls, auditoriums and similar other public places. Furthermore, the rapid scanning decreases the load on testing laboratories for infectious disease by filtering out suspected cases at a fast rate. The individuals with infectious disease can then be referred to testing laboratory or hospitals for further investigation that is more detailed and can be performed on the individual for infectious diseases, specifically COVID-19. Various embodiments described herein generally relate to methods and systems for detecting infectious diseases such as influenza and COVID-19 in an individual, a group, a community, or a city.

### SUMMARY OF THE INVENTION

The present invention provides a novel method and system of infectious disease surveillance. The methods and systems utilize physical scanning of an individual to assess the likelihood of that the individual is suffering from an infectious disease. Additionally, methods and systems for infectious disease surveillance access other parameters such as the medical tests, patient reports, thermal scanning and other external factors to identify the infected cases and carrier cases in large populations. The methods and systems of infectious disease surveillance provide a rapid infectious disease scanning method that can be deployed at public places for identifying individuals/patients having infectious disease or acting as a carrier of the infectious disease.

A computer implemented method and a computer readable medium for rapid scanning and surveillance of an infectious disease in an individual is disclosed. The computer implemented. method receives one or snore parameters as an input from one or more input device. Each of the one or more parameters associated with the individual is extracted by a parameter analysis module. The one or more parameters may be associated with one or more features. In embodiments, the RGB image may be a parameter and different blocks of the RGB image may represent one or more features. In one implementation, the representation of different parts of the individuals face such as nose, eyes, lips are used as features. The computer implemented method further analyze the one or more parameters and associated one or more features by using artificial intelligence algorithms. The artificial intelligence algorithms are stored in the analytical model database. The analytical model database has a test dataset for training one or more analytical models for detection of an infectious disease. The developed models are stored in the analytical models associated with the analytical model database. Further, the computer implemented method selects one of the analytical models from the analytical models based on a pre-determined criterion. Subsequently, the selected analytical model is applied to the one or more parameters and the associated features to estimating the likelihood of the individual having the infection within a certain confidence level. In embodiments, the confidence level may be defined either by a user or through hard coded values in the analytical model. Based on the outcome, the individual may be classified as infected or uninfected with the infectious disease. The computer implemented method provides a report related to the presence of the infectious disease in the individual.

In another embodiment, the computer implemented method and computer readable medium for rapid scanning and surveillance of an infectious disease in an individual is disclosed. The one or more parameter may be associated with one or more features. In one embodiment, the RGB image may be a parameter and different blocks of the RGB image may represent one or more features. In one implementation, the representation of different parts of the individuals face such as nose, eyes, lips may be used as features. The computer implemented method further analyzes the one or more parameters and the associated one or more features using artificial intelligence implemented algorithms. The artificial intelligence implemented algorithms are stored in the analytical model database. The analytical model database has test dataset for training one or more analytical models for detection of infectious disease. The developed models are stored in the analytical models. Further, the computer implemented method selects one of the analytical models from the analytical models based on a pre-determined criterion. Subsequently, the selected analytical model is applied to the one or more parameters and the associated features to estimating the likelihood of the individual having the infection within a certain confidence level. The result for each parameter is stored for further analysis. The computer implemented method aggregates the outcome for each parameter and aggregates and stores them in a data storage. Thereafter, an aggregator module accesses the outcome for each parameter and applies another analytical model to detect infection within a certain confidence level to ascertain the likelihood of infection. Based on the outcome, the individual may be classified as infected or uninfected with the infectious disease. The computer implemented method provides a report related to the presence of the infectious disease in the individual.

In embodiments, the confidence level threshold may be defined either by a user or through a hard coded value in the analytical model.

An infectious disease detection system for rapid scanning, surveillance and detection of an infectious disease in an individual is provided. The infectious disease detection system comprising one or more input devices. Each of the input device is associated with at least one parameter. A parameter analysis module associated with one or more input device extract at least one parameter. At least one parameter is passed to a feature analysis module, which extracts from each parameter one or more features. An analytical model database comprises analytical models. The analytical models are developed using the test data stored in the analytical model database and these developed models are stored in the analytical models. An analytical model selector module associated with the analytical models selects one analytical model based on a pre-determined criterion. Finally, a detection module associated with a processor implements the selected analytical model to detect the presence of the infectious disease and to produce a report to provide information whether the individual is affected by the infectious diseases.

In embodiments, the one or more input devices may be a thermal camera, CT scanner, X-ray machine, infrared camera, RGB camera, RGB-IR camera, clinical analyzer, hematology analyzer, temperature sensor, audio/video recorder and analyzer and similar other devices.

In embodiments, one or more parameters associated with one or more input devices may be a thermal image, RGB image, body temperature, CT scan image, hematology report, clinical test report, audio data, video data or audio-video data.

In one embodiment, the parameter is an RGB image of different parts of the face. Each part of the face may represent one or more features.

In certain embodiments, the pre-defined criteria for selecting the analytical model from the repository of analytical models may be based on number and type of parameters, number and type of features, number and type of input devices and/or the characteristics associated with the individuals such as gender, age, medical history and the like.

In other embodiments, the one or more parameters may be audio-video recordings, body temperature or a CT scan of the individual. In some embodiments, at least one parameter is audio video recording of the individual and such audio video recording has one or a ore features, wherein at least one feature is associated with the audio recording and at least one other feature is related to the video recording. In some embodiments, at least one parameter is the CT scan image of the individual which uses CT scan of the lungs as at least one feature.

In another embodiment, the methods and systems of infectious disease surveillance provide a unique way of identification of individuals infected with infectious diseases such as influenza, COVID-19 by using artificial intelligence (AI). The computer implemented artificial intelligence method and system includes one or more input devices. The one or more input devices may comprise at least one of: a thermal camera, an RGB camera, an infrared camera, an RGB-IR camera, a temperature sensor, a CT scanner, a clinical analyzer, an audio recorder, a video recorder, an audio-video recorder and analyzer, and a hematology analyzer.

The method and system of infectious disease detection may use one or more input devices. In embodiments, the input devices may be a thermal camera, a RGB camera, an infrared camera, a RGB-IR camera, a temperature sensor, a CT scanner, a clinical analyzer, an audio recorder, a video recorder or a hematology analyzer and accordingly the parameter provided by input devices may be the thermal image, the RGB image, the body temperature, the CT scan image, the hematology report, the clinical test report, the audio data, the video data or the audio-video data.

### BRIEF DESCRIPTION OF FIGURES

Different embodiments will now be described in detail with reference to the drawings, in which:

FIG. 1A illustrates a block diagram of operating environment of an infectious disease detection system in accordance with an embodiment of the present invention;

FIG. 1B illustrates various components of an infectious disease detection system in accordance with another embodiment of the present invention;

FIG. 1C illustrates architecture of an artificial engine based analytical model implemented over a cloud in yet another embodiment of the invention;

FIG. 1D illustrates a block diagram of an infectious disease detection system over a cloud in yet another embodiment of the invention;

FIG. 2 is a process flowchart of an infectious disease detection system to detect infection level of an individual using an image in an embodiment of the present invention;

FIG. 3 is a process flowchart of an infectious disease detection system to detect infection level of an individual using an image and an audio/video sample in an embodiment of the present invention;

FIG. 4 is a process flowchart of operating an infectious disease detection system for detecting infection and the associated confidence level to detect an infectious disease in an individual in another embodiment of the invention;

FIG. 5A, FIG. 5B an FIG. 5C illustrate different images of face and hands of an individual using a thermal camera for detecting the infection level;

FIG. 6 illustrates the front and back X-ray of the whole body of an individual using a thermal camera for detecting the infection level;

FIG. 7A and FIG. 7B illustrates the eye scan and/or the retina scan of an individual for determining the redness in eye for detecting the infection level;

FIG. 8 illustrates the CT-scan of chest of an individual for detecting the infection level;

FIG. 9A shows the image of CT scan image of the lungs of the individual having COVID-19 infection;

FIG. 9B shows the image of CT scan image of the lungs of the individual with no infection;

FIG. 10A illustrates a thermal image of the upper region of the face for detection of the infectious disease, showing distribution of temperature on and around the nose of an individual without an infection, in an embodiment of the invention;

FIG. 10B illustrates a thermal image of the upper region of the face for detection of the infectious disease, showing distribution of temperature on and around the nose of an individual infected with flu and fever, in an embodiment of the invention;

FIG. 11 illustrates the rapid screening architecture for detection of the infectious disease in an embodiment of the invention;

FIG. 12 illustrates the user interface showing contribution of different parameters of an individual to infection detection in an embodiment of the present invention;

FIG. 13 illustrates the scan of upper body in right orientation for detecting the infected individual using a thermal camera in another embodiment of the invention;

FIG. 14 illustrates a user interface of the infectious disease detection system in an embodiment of the present invention;

FIG. 15 illustrates the different evaluation parameters and outcomes for detection of an infectious disease in an embodiment of the present invention.

FIG. 16 shows the data flow of the infectious disease detection system for tracking and management in an embodiment of the invention.

### DETAILED DESCRIPTION OF THE INVENTION

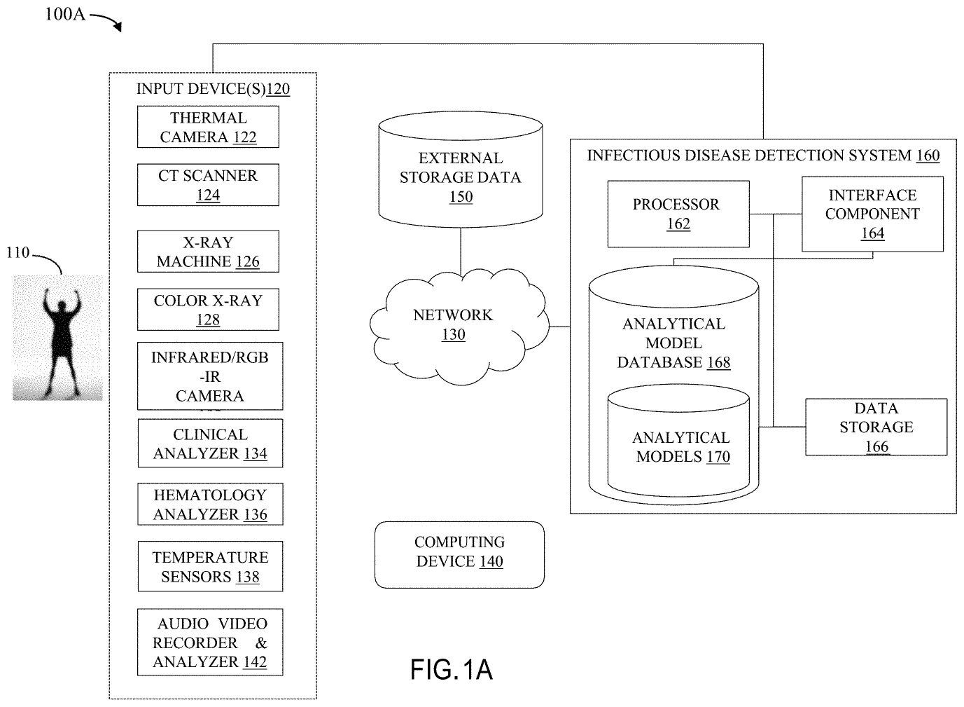

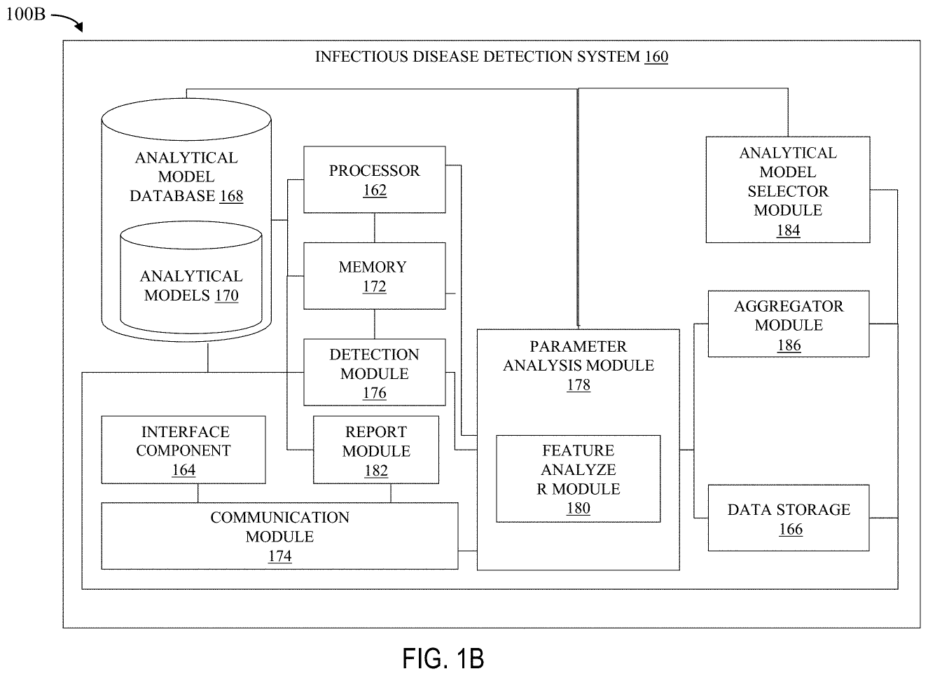

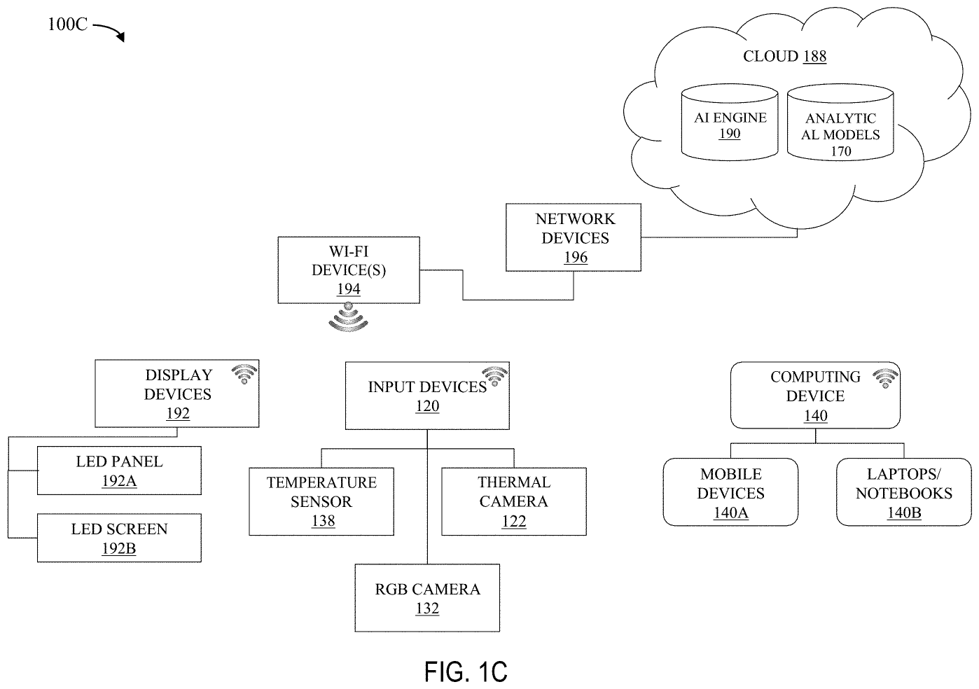

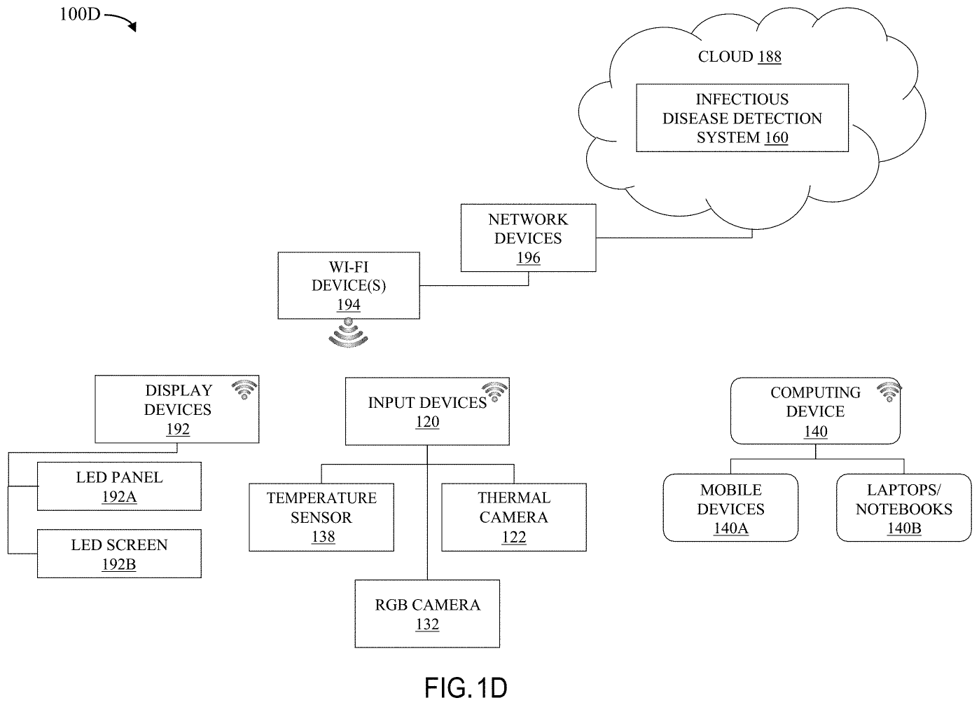

FIG. 1A illustrates the operating environment of an infectious disease detection system for scanning, surveillance and detection of infectious diseases in an embodiment of the present invention. The operating environment 100A includes one or more input devices 120, an external storage database 150, a network 130, at least one computing device 140 and the infectious disease detection system 160 and an individual 110 undergoing the infectious disease test.

The infectious disease detection system 160 includes a processor 162, an interface component 164, and data storage 166 apart from other modules and components. The data storage 166 stores data received from one or more input devices 120, data from one or more computing devices 140 and data received from other sources. In addition, the data storage 166 stores and implementations different artificial intelligence algorithms and deep learning algorithms. The data storage 166 is connected directly to the processor 162 and the interface components 164. The interface component 164 provides an interface for data exchange with the external devices such as router, servers, medical databases, insurance database, testing labs, and other interested parties such as but not limited to hospitals, doctors, general practitioners that are provide information and data related to the infectious disease(s) associated with the individual 110.

The infectious disease detection system 160 also includes an analytical model database 168 and a repository storing multiple analytical models 170. The analytical model database 168 includes several algorithms, training data set, rules for development of analytical models. Each algorithm is selected based on multiple factors and depending upon the data available in the database, training data, type of data and other variables such as number of input devices 120 to be utilized for detection of the infectious disease(s). For example, the type of data may be an image data, a voice data, clinical test data, and/or a body temperature of the individual or some other type of data.

In some embodiments, the algorithms stored in the analytical model database 168 are implemented to develop or update the analytical model(s) using the training data received from the data storage 166. The updated or developed analytical models are stored in the analytical models 170.

In some embodiments, the analytical model database 168 and the analytical models 170 may be stored in the data storage 166. In alternate embodiment, the analytical model database 168 and the analytical models 170 can be stored in the external data storage 150 accessible via the network 130. In this implementation of the algorithm enabled for deep learning is implemented on an external server, a cloud, a distributed system or a central repository having its own memory and. processor and accessible through a network 130.

The infectious disease detection system 160 can communicate with the external storage data 150 and at least one computing device 140 through the network 130. Additionally, the infectious disease detection system 160 can also communicate with one or more input devices 120 via the network 130.

The at least one computing device 140 may be a desktop computer, a smart phone, a tablet, a palm held device having a processor and memory to communicate with the infectious disease detection system 160 either through a wireless or a wired connection. 100521 The one or more input devices 120 can be a thermal camera 122, a CT scanner 124, a X-ray machine 126, an infrared camera or RGB-IR camera 132, a clinical analyzer 134, a hematology analyzer 136, a temperature sensor 138, and an audio video recorder and analyzer 142 and some other type of medical devices for example antigen test kits, virology detection devices, etc.

In different embodiments, the one or more input devices 120 associated with the infectious disease detection system 160 can be at least one of the thermal camera 122, the CT scanner 124, the X-ray machine 126, the color X-ray machine 128, the infrared camera 132 or RGB-IR camera, the clinical analyzer 134, the hematology analyzer 136, temperature sensors 138 and the audio video recorder and analyzer 142 or some other input device such as but not limited to a keyboard, a bar code scanner, an audio recorder, a video recorder, a mobile device with a scanner, a mobile device with a camera, a mobile device with a one or more sensors or some other type of input device. For example, in the one of the embodiments, the input devices 120 connected to the infectious disease detection system 160 are thermal camera 122, the CT scanner 124, the infrared camera or RGB-IR camera 132, the temperature sensors 138 and the audio video recorder and analyzer 142.

In some embodiments, the one or more input devices 120 can be directly coupled with the interface component 164 so as to form an integrated part of the infectious disease detection system 160.

In different embodiments, the infectious disease detection system 160 can be implemented in a distributed system having distributed components, on one or more servers, distributed architecture over a distributed networked servers or systems, a cloud computing environment, a mobile electronic computing device having one or more input interface to access one or nore input devices 120 such as but not limited the thermal camera 122, the CT scanner 124, the X-ray machine 126, the color X-ray machine 128, the infrared camera 132 or RGB-IR camera, the clinical analyzer 134, the hematology analyzer 136, temperature sensors 138 and the audio video recorder and analyzer 142 or some other type of input device.

The processor 162 may be any general or special purpose processor, a microcontroller or digital signal processor that provides sufficient processing power depending on the configuration, purposes and requirements of the infectious disease detection system 160.

In some embodiments, the processor 162 can be a multi-core, hyper threaded processor, which is configured to perform different dedicated tasks. The processor 162 controls the operation and data processing within the infectious disease detection system 160. For example, the processor 162 can receive a set of data associated with an individual 110 from a one or more input devices 120 either through the interface component 164 or directly or through the data storage 166 and process the data in accordance with the methods and processes disclosed herein and elsewhere to determine whether the individual 110 is infected with an infectious disease.

In preferred embodiments, the infectious disease detection system 160 is implemented for scanning, surveillance and detection of COVID-19.

In some embodiments, the multiple analytical models 170 associated with the infectious disease detection system 160 for scanning of infectious diseases associated with the individual 110 may include one or more algorithms such as but not limited to supervised learning, unsupervised learning, semi-supervised learning, reinforcement learning, self-learning, feature learning, sparse dictionary learning, anomaly detection, robot learning, federated learning and association rules or may implement any combination of these algorithm.

In some embodiments, the multiple analytical models 170 associated with the infectious disease detection system 160 for investigation of infectious diseases associated with the individual 110 may include one or more algorithms such as but not limited to artificial neural networks, deep learning, multilayer artificial neural network, decision trees, support vector machines, regression analysis, Bayesian networks, genetic algorithms or may implement any combination of to these learning techniques.

FIG. 1B illustrates various components of an infectious disease detection system in another embodiment of the present invention. The infectious disease detection system 160 include the processor 162 coupled to a memory 172, a detection module 176, a report module 182, a communication module 174, a parameter analysis module 178, which includes a feature analyzer module 180. Furthermore, the infectious disease detection system 160 includes an analytical model selector module 184, an aggregator module 186, the interface component 164, the data storage 166, and the analytical model database 168 having the analytical models 170. 100B shows an exemplary variation of the infectious disease detection system 160 with additional modules; however, in other variations there may include additional/fewer modules than shown in FIG. 1B.

The memory 172 may be a RAM, a ROM, a tape drive, a flash memory, an EPROM or some other type of storage medium.

### Parameter Detection and Processing

The input data/image/audio/video from one or more input devices 120 is passed a one or more parameters. The one or more parameters are received by the interface component 164 or the communication module 174, which then passes the one or more parameters to the parameter analysis module 178. The parameter analysis module 178 selects at least one parameter for detection of infectious disease. The selected at least one parameters is passed to the feature analyzer module 180, which may extract features, which may be utilized by the analytical models 170 for detection of the infectious diseases in the individual 110.

The one or more parameters associated with one or more input devices 120 may be a temperature data received from the temperature sensor 138. Likewise, the one or more parameters received from the with one or more input devices 120 may be a CT scan image received from the CT scanner 124. Similarly, the one or more parameters received from the with one or more input devices 120 may be a RGB image or RGB image data received from the RGB-Infrared camera 132. Furthermore, the one or more parameters received from the one or more input devices 120 may be an audio/video data received from the Audio video recorder and analyzer 142. Similarly, the one or more input devices 120 may provide image data. CT scan data, hematology report, RGB image data, X-ray report, them al image or some other type of data associated with the input devices 120.

In one variation, the at least one parameter selected by the parameter analysis module 178 may be processed to extract one or more characteristics. Each parameter may have one or more associated characteristics. A characteristic analyzer module (not shown in figure) extracts different characteristics associated with one or more parameters. In some embodiments, the characteristics associated with one or more parameters may be utilized by the analytical model 170 to determine the presence of infectious disease in the individual 110.

In one implementation the features and characteristics associated with one or more parameters received from one or more input devices 120 may be utilized for detecting the infectious disease using the analytical model 170.

### Feature Detection

The detection module 176 associated with the parameter analysis module 178. The parameter analysis module 178 provides one or more parameters associated with the data received from one or more input devices 120 to the detection module 176. For example, when an image file is received from the RGB camera, the RGB image may be divided one or more features blocks, for example, a 9×9 matrix. Each block may refer to at least one feature. The one or more parameters along with one or more features may be analyzed by the detection module 176 using at least one analytical model to detect the presence of infectious disease. Likewise, thermal camera may capture the heat distribution of the different body parts of the individual 110 and accordingly apply analytical model for determination of the infectious disease. As explained earlier the thermal image maybe divided into blocks and each block may represent one or more feature, which is analyzed by the detection module 176. Similarly, the CT scanner may capture CT scan image(s). Features may be extracted from the associated parameter; the one or more feature extracted from the CT scan images is provided to analytical models 170 for determination of infectious disease(s). Likewise, the audio video recorder and analyzer 142 may extract features audio video clip, which are applied to the analytical model 170 to determine the presence of infectious disease.

### System Operations

Referring back to FIG. 1B, the infectious disease detection system 160 receives input data/image/audio/video from one or more input devices 120. The data/image/audio/video is received at the interface component 164 either directly or through a wired or wireless network. The received data/image/audio/video is passed to the communication module 174, which passes the received data/image/audio/video to the parameter analysis module 178. The parameter analysis module 178 analysis one or more received parameters. The feature analysis module 180 associated with the parameter analysis module 178 then evaluates and extracts on or more features from the received data/image/audio/video.

In some embodiments, the feature extraction module 180 determines if any further characterization of the received data/image/audio/video can be performed and if so, then received data is passed to the characteristic analyzer module 182 for extraction of one or more characteristics. This step is optional and may not be performed in certain embodiments.

Finally, a processed data comprising one or more parameters and one or more features is passed to the data storage 166 and the analytical model database 168 for storage. In addition, the processed data is passed to aggregator module 186.

In certain embodiments, the processed data is passed to the detection module 176. The detection module 176 access the analytical model selector module 184 to select an analytical model based on a pre-determined criterion. In embodiments, the pre-determined criteria for selection of an analytical model may be based on parameter type, feature type, feature characteristics, gender, age, medical history or some other variable associated with the individual 110. Once the analytical model selector module 184 has selected an analytical model to be applied for detection of the infectious disease from the analytical models 170, the process of analysis of infectious disease using the selected analytical model is initiated.

The detection module 176 uses the processed data stored in the data storage 166 and the selected analytical model to detect the presence of an infectious disease in the individual 110. The detection module 176 passes the result related to the individual 110 to the report module 182. The report module provides the outcome either on the user interface or in tangible formats such as paper or e-report. In some embodiments, the report module may provide the individual 110 with the severity of the infectious disease along with suggestions for further test. In some embodiments, the infectious disease detection system 160 may inform the general practitioner, the hospital, the health department about the outcome of result for further action.

The analytical model database 168 uses the data to train/retrain and update the stored analytical models 170 with the new data. In preferred embodiments, the retraining of the machine learning algorithms may be supervised by an operator based on data received after further investigation. Alternatively, the analytical models 170 may be updated, when a specified rule defined in the analytical model database 168 is triggered. In another variation, the analytical model database 168 may have a rule based engine to update the analytical model database 170.

In some embodiments, the suggestion may be related to performing additional test if the selected analytical model produces a positive result for a probability of an infectious disease. In some other embodiments, the suggestion may be related asking the individual 100 to isolate due to high probability of presence of infectious disease.

### Ensemble Learning-Bagging and Boosting

In another embodiment, the infectious disease detection system 160 may implement bagging and boosting for a strong and accurate prediction. In this variation, the infectious disease detection system 160 includes the aggregator module 186 along with the analytical model selector module 184 for detection of the infectious disease.

The detection nodule 176 receives one or more parameters along with one or more features from the parameter analysis module 178 and the feature analysis module 108. For one or more parameter received from one input device 120, the detection module 176 performs the detection of the infectious disease. The result may be stored in the data storage 166. Likewise, for each received parameter the process for detection of the infectious disease is performed. The result for each of the parameters is stored in the data storage 166. Once all the results have been aggregated, the results are passed to the aggregator module 186. The aggregator module 186 then applies a different analytical model to the aggregated results. The outcome provides a strong and accurate prediction that the individual 110 is suffering from an infection disease.

To illustrate with an example, the individual 110 is scanned by one or more input devises 120. The input devices may be the RGB-IR camera 132 providing a RGB image, CT scanner 124 providing a CT scan report, and audio video recorder and analyzer 142 providing an audio-video recording as corresponding parameters. Each parameter such as the RGB image may be analyzed by the selected analytical model to predict if the individual 110 is suffering from an infectious disease. Likewise, the CT scan report may be analyzed by the selected analytical model to predict if the individual 110 is suffering from an infectious disease. Similarly, the audio-video recording may be analyzed by the selected analytical model to predict if the individual 110 is suffering from an infectious disease. The outcomes corresponding to each parameter, that is, the RGB image, the CT scan report, and the audio-video recording may be stored and further analyzed using a different analytical model to arrive at the final outcome, that is, whether the individual is suffering for an infectious disease.

In some embodiments, the select analytical model for detection of the infectious disease for each parameter at the first level analysis and the analytical model applied to the outcome of each parameter to arrive at final value may be different or same. In other embodiments, the selected analytical model for detection of the infectious disease for each parameter may be same or different.

In one variation, after the analysis of each parameter associated with the one or more input devices 120, the aggregator module 186 may apply discriminate or weighted analysis to the outcome of each parameter to identify highly significant variables. The highly significant variables may be provided as an input to the selected analytical model from the analytical models 170. The final outcome would a report of presence of infectious disease in the individual 110.

### Confidence Level

The infectious disease detection system 160 can exclude some of the likelihoods based on the outcome of the analytical model used for detection of the infectious disease. When one or more parameters associated with one or more input devices 120 are utilized for detection of the infectious disease, the outcome are quantified based on a confidence level. A confidence level refers to the percentage of all possible samples that can be expected to include the true population parameter. The infectious disease detection system 160 can determine whether a confidence level associated with infection likelihood satisfies a confidence threshold. The confidence threshold corresponds to a minimum confidence level necessary to confirm infection in the individual 110. When the infectious disease detection system 160 determines that the confidence threshold is not satisfied, the infectious disease detection system 160 can eliminate that infection likelihood.

In some embodiments, the confidence level and the confidence threshold may be set by a user. The user may set the confidence level and the confidence threshold based on the type of analytical model, the number of parameters associated with the input devices 120, the training data some other variable associated with the analytical model.

### Training Data Set

The analytical model database 168 may include training data set for training and developing analytical models. The pre-validated training data set may be stored in the analytical model database, which may be updated through the network 130 from external sources, for example, other computing devices 140 or external databases from research laboratories 150. This results in continuous refined of the analytical models for better prediction. The analytical models are stored in the analytical models 170.

In some embodiments, the training data set may be stored in the data storage 166. In some other embodiments, the operator may intervene to train the analytical model database 168. When the analytical models 170 are updated and refined, the infectious disease detection system 160 may update the XML scheme of the analytical model selector module 184 related to selection of different analytical models during prediction. As discussed, the XML scheme may be updated based by the user in one embodiment.

In one embodiment, the method of detecting the individual 110 infected with infectious disease involves operating the processor 162 to receive a set of training data. The training data may include data such as but not limited to images, videos, clinical data, medical test report, RGB image for face, lungs, nose, eyes and forehead and CT scan of whole body scan for one or more individuals. The method uses the training data set such as the images, videos, clinical data, medical test report and RGB image for face, lungs, nose, eyes and forehead to create or develop an infection detection analytical model. The method then applies the trained analytical model to perform the rapid scanning of one or more individual 110 to detect the presence of the infectious disease.

In another embodiment, the method of detecting the individual 110 infected with infectious disease involves operating the processor 162 to receive a set of training data. The set of training data includes only images, which can be thermal images, RGB images or RGB-infrared images. In one embodiment, the received image data may be in form of a heat map of the different sections of the body or an RGB image. For example, the thermal image may provide the heat map of the face taken at different angles such as the front view, side views that is left view and right view of the human face and/or the image of the forehead. The heat map of the forehead shows the body temperature of the individual 110. In another embodiment, the RGB image can be of eyes that show the redness in each eye, or a heat map of the temperature between eye pupils. In yet another implementation, the image can be an X-ray of the chest, thermal image showing heat map of the lungs, ultrasound of the lungs. In another embodiment, the image could be CT scan of the upper body, specifically of the chest or even other parts of the body. In yet another embodiment, the training data may include thermal images, RGB images, RGB-infrared images or X-ray image or any combination of these images.

In another embodiment, the method of detecting the individual 110 infected with infectious disease involves operating the processor 162 to receive a set of training data. The set of training data includes clinical data and/or pathological data/reports such as but not limited to liver function test, kidney function test, creatinine level received from or some other clinical or pathological data. In another embodiment, the training data may be hematology data such as but not limited to RBC counts, WBC counts, eosinophils and basophiles. In another embodiment, the training data may be pulse data and SpO₂ data. In yet another embodiment, the training data may be a combination of clinical data, hematology data, pulse data and SpO₂ data.

In some embodiments, the method of detecting the individual 110 infected with infectious disease involves operating the processor 162 to receive a set of training data. The training data may be related to whole body scan. The different body parts of interest that show symptoms such as tip of nose, color of eyes, hands, forehead and face. In another embodiment, the training data set may include capturing feedback of the individual using an audio device and asking the individual to provide Yes/No feedback related to taste, smell, and hunger. In yet another embodiment, the training data may include RGB image for face, X-ray or CT scan of lungs or chest, whole body scan. In yet another embodiment, the training data set may include a combination of the whole body scan and feedback related to taste, smell, and hunger.

In some embodiments, the method of detecting the individual 110 infected with infectious disease involves operating the processor 162 to receive a set of training data. The training data may include one or more or any combination of parameters such as images, video, clinical data, medical test report and RGB image for face, CT scan or X-ray or thermal image of lungs, whole body scan or some other parameters related to the individual 110.

In some embodiments, developing an infection detection analytical model involves applying at least one of the machine learning algorithms or artificial intelligence algorithms. The training data set may either be stored in the analytical model database 168 or the data storage 166. The analytical model created using the training data is stored in the analytical models 170 associated with the analytical model database 168. In different embodiments, one or more analytical models may be developed and stored in the analytical models 170. In one variation, a pattern recognition algorithm may be applied to the training set data comprising images, video, clinical data, medical test report and RGB facial images and determining the confidence level to predict the individual being infected by an infectious disease such as COVID-19. In embodiments, the pattern recognition algorithm includes an algorithm based on at least one of Nearest Neighbor, K-Nearest Neighbors, Support Vector Machines, Naive Bayesian, Decision Trees, Random Forests, Logistic Regression, and/or Linear Discriminant Analysis.

The training set data may include pre-validated data of individuals suffering from infectious disease and free from infectious disease. In some embodiments, the pre-validated data of individuals may be categorized based on the severity of the infection and utilized for training the artificial intelligence and/or machine learning algorithms to predict and categorize the severity of infection.

In some embodiments, one or more analytical models for predicting infection or level of infection may be trained using the training dataset with one parameter per input device. For example, the parameter may be thermal distribution of temperature or a heat map of different parts of the face. Likewise, one or model analytical model may be developed for each parameter. Subsequently, another analytical model may be developed a training dataset that comprises outcome of each parameter as an input.

In some embodiments, there is provided a non-transitory computer-readable medium having executable instructions stored in the memory 172 that when executed by the processor 162 to perform the steps of scanning and detecting the infectious disease in the individual 110. The method and system receive the training dataset that includes images, video, clinical data, medical test report and RGB image for face, hands, forehead and eyes, CT scan, whole body scan from one or more individuals. The method and system further associate each training dataset comprising images, video, clinical data, medical test report and RGB facial images with infected level. Subsequently, an infection detection analytical model based on the set of training data comprising images, video, clinical data, medical test report and RGB facial images is developed for predicting the infectious disease in the individual 110. The method and system then apply the trained infection detection analytical model to perform the rapid scanning of individuals for being infected by an infectious disease such as COVID-19.

FIG. 1C illustrates architecture of an artificial engine based analytical models implemented over a cloud in yet another embodiment of the invention. The cloud based artificial intelligence architecture 100C includes a cloud 188, an artificial intelligence engine 190 coupled with analytical models 170. In some embodiments, the artificial intelligence engine 190 includes the detection module 176, the analytical model database 168, the parameter analysis module 178, the feature analysis module 178, the analytical model selector module 184, the aggregator module 186 and the data storage module 166. In another implementation, the artificial intelligence engine 190 includes the detection module 176, the analytical model database 168, the parameter analysis module 178 and the feature analysis module 178. In yet another implementation, the artificial intelligence engine 190 includes the detection module 176, the analytical model selector module 184, the aggregator module 186 and the data storage module 166.

The cloud 188 may be interconnected with one or more network devices 196. The one or more network devices 196 may include devices such as switches, routers, access points and wireless modems. The one or more network devices 196 provide a wireless infrastructure for connecting the cloud 188 with one or more display devices 192, one or more input devices 120 and one or more computing devices 140.

In addition, the cloud based artificial intelligence architecture 100C further includes one or more display devices 192 such as but not limited to LED panels 192A, large LED screens 192B for public displays. Additionally, the one or more input devices 120 in this implementation include the temperature sensor 138, the thermal camera 122, and the RGB camera 132. In other implementation, the one or more input devices 120 may include other devices as described. The

The one or more display devices 192 may include LED panels 192A, LED screens 192B or some other type of display devices.

The one or more computing devices 140 may include mobile devices 140A, Laptop/notebooks 140B or some other type of computing devices.

FIG. 1D illustrates a block diagram of an infectious disease detection system over a cloud in yet another embodiment of the invention. In this implementation, the infectious disease detection system 160 may be implemented over the cloud 188. In variations of this embodiment, the infectious disease detection system 160 may have fewer or additional modules. For example, the processor 162 and memory 172 may be absent as the processor 162 and the memory 172 is available in the cloud 188 infrastructure.

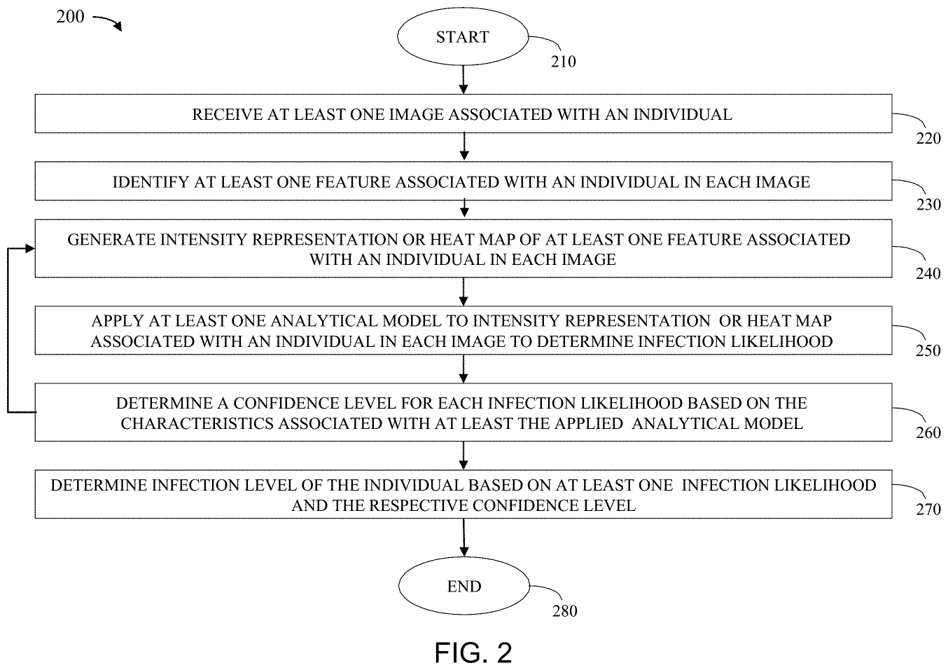

FIG. 2, in an exemplary embodiment, illustrates the process 200 of detecting the infectious disease by the infectious disease detection system 160. The infectious disease detection system 160 can receive image or other type of data from one or more input devices 120 associated with the individual 110 as one or more parameters. The process 200 of detection of infectious disease by the infectious disease detection system 160 is initiated at 210. During initiation of the process 200 receives image or other type of data associated with the individual 110 for rapid scanning of infectious disease, for example, COVID-19. At step 220, the process 200 identifies at least one feature associated with at least one parameter received from one or more input devices 120.

In one embodiment, the infectious disease detection system 160 may evaluate the type of input and the parameter from one or more input devices 120. For example, the input received as parameter may be a thermal image. In another embodiment, the infectious disease detection system 160 may evaluate the input received as parameter from one or more input devices 120 as a RGB images of individual's face. The RGB image of the individual 110 may include RGB image of the hand, the face, the forehead and the eyes. In some embodiment, the infectious disease detection system 160 may evaluate input received as parameter from one or more input devices 120 in an ordered sequence, for example, RGB image (highest priority), CT scan image (second priority) and audio and/or video image (third priority). Alternatively, the infectious disease detection system 160 may evaluate input received as parameter from one or more input devices 120 in a pre-defined order as provided by the user. This selection is based on selection of parameters that provide high accuracy in prediction.

At step 230, the one or more features associated with one or more parameters are identified by the infectious disease detection system 160 in each image. In embodiments, the feature may relate to a property or characteristic identifiable in the image. For example, the feature can relate to an intensity distribution within an image, and/or a region of interest, and/or color intensity corresponding red, green and blue. The identification of property and/or features automatically done by the model or may be provided by the user, wherein the user may define the sequence of the order of input device(s) 120.

In one implementation, the infectious disease detection system 160 can apply at least one analytical model to the image or other type of data for identifying the region of interest, such as eyes, hands or face or other portion of the body. In addition, in some embodiment, the analytical model can also determine the number of inputs that might be required for predicting the infection level in the individual 110. In some embodiments, at least one analytical model may be applied to one or more features for different body parts; the results are stored in the data storage 166. For example, analytical model X may be applied to the forehead while analytical model Y may be applied to eyes.

In some embodiments, the infectious disease detection system 160 can apply different image analysis techniques to the image, such as image preprocessing to enhance relevant pixel information, for example, and/or image segmentation to focus on the regions of interest. Further, to identify one or more body parts to which each image relates, the infectious disease detection system 160 can apply the feature extraction models or feature analytical model to determine the body part has the highest associated confidence level. Alternatively, the infectious disease detection system 160 can decide which of the images from one or more input devices 120 have the associated confidence level for prediction of the infectious disease. To assist with the feature extraction process, the analytical models 170 associated with implementing feature extraction algorithms may preprocess the image/image data to assist with identifying one or more features. For example, the infectious disease detection system 160 implement image processing algorithms, which can apply different transformations to the received image for better accuracy such as but not limited to scaling, rotation, grey-scaling, cropping, or some other image transformation techniques. In some embodiments, the preprocessing can normalize image/image data without modifying different ranges of intensities of the image data for consistency. Furthermore, the infectious disease detection system 160 can also reduce noise in the image data to improve detection accuracy.

In some embodiments, one or more analytical models 170 associated with feature extraction algorithms are also referred as feature analytical models. Likewise, one or more analytical models 170 associated with image transformation algorithms are also referred as image processing analytical models.

The infectious disease detection system 160 can also generate the feature analytical models based on a set of features associated with training image dataset of one or more individuals 110. For example, the infectious disease detection system 160 can generate the feature analytical models using convolutional neural networks (CNNs). Each training image dataset can be associated, or labeled or tagged, with one or more features and the infectious disease detection system 160 can analyze each labeled training image dataset or the specific region of interest to develop the feature analytical model for the one or more tagged or labeled features. The infectious disease detection system 160 can store the feature analytical model in the data storage 166. In some embodiments, the infectious disease detection system 160 can continue to update the feature analytical model with new training dataset.

For example, when the infectious disease detection system 160 receives multiple images from one or more input devices 120, the infectious disease detection system 160 can identify the feature(s) by applying the feature analytical models stored in the analytical models 170 or the data storage 166 to determine which features and/or which input devices need to be prioritized and also determine the prioritization sequence. From applying the feature analytical models to the multiple images, the infectious disease detection system 160 can determine: which images are more likely related to the eyes, which images are more likely related to the face, and which images are more likely related to the forehead of the individual 110.

In some embodiments, the infectious disease detection system 160 can apply various pattern recognition algorithms stored in the analytical model 170 or the data storage 166 to automatically identify one or more features represented by the multiple images received from one or more input device 120. Example of the pattern recognition algorithms implemented as analytical models and stored in analytical model 170 include but are not limited to techniques based on histograms of gradient, local binary patterns and/or harr like features. For example, the infectious disease detection system 160 can generate a local binary pattern based on the received image from one or more input devices 120. The features extracted from the local binary pattern can be stored in as feature vectors. In some embodiments, the vector features may be normalized for better prediction accuracy.

In some embodiments, the infectious disease detection system 160 can extract a portion of the received image data for analysis and further processing. For example, the infectious disease detection system 160 can extract the portion of the image data related to the face and eyes of the individual 110.

Referring to step 240, the infectious disease detection system 160 generates an intensity representation for each feature identified at the step 230. In another embodiment, the infectious disease detection system 160 may generate the heat map for different regions of each of the body parts, for example, eyes, hands, and face or other parts, which are received as parameters. The intensity representation represents the intensity at each pixel of the received image data. For infrared images of the face, the color of each pixel is associated with the heat intensity of the face of the individual 110. The heat intensity map for each pixel of the individual 110 changes with the infection level due to changes in the breathing rate, the lung capacity, and other factor associated with human body. In one embodiment, the intensity representations and the variability in the heat distribution of the eyes, the nose and the forehead at the different infection levels can be used by the analytical model to predict level of infection levels.

In some embodiments, the infectious disease detection system 160 can generate the intensity representation for a portion of the image. For example, the infectious disease detection system 160 may generate a histogram to represent intensity values of a particular portion of the image identified as the area of interest.

At step 250, the infectious disease detection system 160 applies at least one analytical model to the intensity representation to determine likelihood of the infection in the individual 110. In some embodiments, the infectious disease detection system 160 applies at least one analytical model to the intensity representation to determine the level of infection.

The analytical models 170 relate identified one or more features along with the associated intensity representation received as parameter from one or more input device 120 to the infection level detected in the individual 110. Different analytical models can be developed for different features. For example, an analytical model can be developed for images related to the side profile of the head that are associated with high infection. In another example, an analytical model can be developed for images related to the side profile of the head. In yet another example, an analytical model can be developed for images of the hands for the individual 110 showing mild infection. Alternatively, in another example, a different analytical model can be developed for images of the hand for the individual 110 showing no infection.

In some embodiments, the infectious disease detection system 160 may develop analytical models using the training dataset images associated with individuals 110 that are non—infected with any infectious disease. Alternatively, the infectious disease detection system 160 may develop analytical models using the training dataset images associated individuals 110 associated with high level of infection. Any images associated with individuals 110 that does not fit well within the prediction of high infection to no infection may classified under low level of infection or medium level of infection or vice versa.

The infectious disease detection system 160 applies the analytical model to each intensity representation to determine how closely the features corresponding to the intensity representation align with the features represented by the analytical model. For example, the infection likelihood can correspond to a numerical value (e.g., a percentage) indicating how well each intensity representation fits with respect to the applied analytical model. In some embodiments, the infectious disease detection system 160 can generate a binary determination for the infection likelihood to evaluate if the intensity representation fits or does not fit the binary representation output of the analytical model.

In some embodiments, the infectious disease detection system 160 can generate the analytical model based on a set of training images, which may correspond to either no infection or high infection of the individual 110. The set of training images, which are aggregated from one or more input sources 120 and can be stored in the data storage 166 or alternatively in the analytical model database 170. The set of training images can include one or more images associated with different individuals, and each image can be associated with an infection level ranging from no infection, low infection, medium infection or high infection. The infectious disease detection system 160 can then generate an analytical model based on the set of training images and the infection level associated with the set of training images. For example, the infectious disease detection system 160 can generate the analytical model by applying a pattern recognition algorithm to the set of training images. The different categories of infection levels, that is, no infection, low infection, medium infection or high infection are associated by the pattern recognition analytical model to establish patterns and/or relationship with the features identified in the set of training images and the individual 110 under prediction. The outcome may be a report showing the level of infection. In some embodiments, the analytical model may implement multiple pattern recognition algorithms for detection of the infectious disease associated with the individual 110.

In different embodiments, the pattern recognition algorithms implemented for prediction of the infectious disease associated with the individual 110 may include, but are not limited to, techniques based on nearest neighbor, k-nearest neighbors, support vector machines, naive Bayesian, decision trees, random forests, logistic regression, and/or Linear Discriminant Analysis (LDA). With different pattern recognition algorithms implemented in the analytical models 170 different aspects of the intensity representation can be analyzed. For example, the infectious disease detection system 160 can apply the logistic regression technique to establish linear relationships between the identified features. In another example, the infectious disease detection system 160 can apply the random forest technique to develop a set of rules based on the identified features.

At step 250 of the process 200, the infectious disease detection system 160 determines a confidence level associated with infection likelihood based on characteristics and/or one or more features of the received parameter, for example, image or audio data. These characteristics and/or features are applied to at least one selected analytical model selected from the one or more analytical models 170 for making prediction of the individual 110 having an infectious disease.

The output result of the infection level provided by the infectious disease detection system 160 may vary based the type of parameters received form one or more input devices 120. In addition, the output result may vary based on the data received by the infectious disease detection system 160 and/or the selected analytical model from the analytical models 170. For example, the CT scanner 124 may provide better quality of parameters and features to accurately detect the infection level in lungs of the individual 110 with a higher confidence level. In another example, the temperature sensor may only provide body temperature as a parameter and the prediction may not be as accurate as the result obtained with parameter and feature received from the CT scan data. In addition, some of the analytical models may be more reliable in predicting the infectious disease as these analytical models can provide and generate output with higher confidence level.

Another variable that can affect the outcome of the prediction of the infectious disease detection system 160 is the image quality. In some embodiments, the infectious disease detection system 160 may employ and implement image quality improvement algorithms, when the image quality is below a predefined quality threshold. In these embodiments, the infectious disease detection system 160 can generate a quality indicator to indicate that the image quality satisfies the predefined quality threshold causing a positive effect on the confidence levels associated with the prediction of the infectious disease. The quality threshold indicates a minimal resolution required for the image quality to be satisfactory for prediction of the infectious disease.

The infectious disease detection system 160 employs a novel way to improve the quality of image(s) received as parameters and associated with the prediction of the infectious disease. If the image quality is found below the predefined threshold level, the infectious disease detection system 160 can improve the image by applying image improvement algorithms above the predefined threshold level. In case, the improvement of the received image(s) is not possible, the infectious disease detection system 160 extract the point of interest in one or more images and evaluate if the quality of section of images that are points of interest can be improved. If so, the infectious disease detection system 160 performs the detection of the infectious disease using one or more analytical models 170. If this fails, the infectious disease detection system 160 alerts the user or the operator about the bad quality of image.

The infectious disease detection system 160 may consider the view or perspective of the image, when detecting the infectious disease of the individual 110. For example, the infectious disease detection system 160 may receive the front view of the forehead and consider it to be less reliable than the side view of the forehead. The image reliability indicator, which determines the reliability, can vary depending on the type of view of the image. The image reliability indicator can have a binary value (‘0’ for low reliability and ‘1’ for high reliability) or a decimal numerical value. The infectious disease detection system 160 can then factor the image reliability indicator into the confidence level for each of the infection likelihood, which is associated with one or more parameters.

In some embodiments, the infectious disease detection system 160 can consider the type of features when defining the infection of the individual 110. For example, while comparing the intensity representation associated with the side profile of the head and the intensity representation associated with the eyes and hands; the intensity representation associated with the eyes and hands can provide information, which is more accurate and valuable. The infectious disease detection system 160 can assign a higher feature reliability indicator to the intensity representation corresponding to the eyes and hands. The feature reliability indicator can be a binary value (‘0’ for low reliability and ‘1’ for high reliability) or a decimal numerical value. The infectious disease detection system 160 can then determine the confidence level for infection likelihood based on the feature reliability indicator.

The infectious disease detection system 160 may consider the type of analytical model used for detecting the level of infection. Some of the analytical models may be more accurate in certain conditions, while the other analytical models have high accuracy under different conditions. In this aspect, the infectious disease detection system 160 can assign a model reliability indicator to each analytical model and vary the confidence level for each of the infection likelihood. based on the model reliability indicator. The model reliability indicator can be a binary value (‘0’ for low reliability and ‘1’ for high reliability) or a numerical value.

In some embodiments, the steps 240, 250 and 260 of the process 220 may be performed iteratively by the infectious disease detection system 160 for each feature and parameters associated with one or more input devices 120 and identified at the step 230.

At step 270 of the process 200, the infectious disease detection system 160 defines the infection level of the individual 110 based on at least one of the infection likelihoods and the respective confidence level.

In embodiments, the infection level of the individual 110 can be represented by an infection level indicator such as no infection, low infection, medium infection, and high infection indicating how infected the individual is with the infectious disease such as COVID-19. For example, the infection indicator can be a text indicator, such as no infection, low infection, medium infection, and high infection.

In some embodiments, the infectious disease detection system 160 can define the infection of the individual 110 by taking an average of each of the infection likelihood weighted with the associated confidence level. In some embodiments, the infectious disease detection system 160 can determine the infection likelihood based on multiple indicated values.

In some embodiments, the infectious disease detection system 160 can exclude some of the infection likelihoods. For example, the infectious disease detection system 160 can determine whether a confidence level associated with infection likelihood satisfies a confidence threshold. The confidence threshold corresponds to a minimum confidence level necessary to confirm infection in the individual 110. When the infectious disease detection system 160 determines that the confidence threshold is not satisfied, the infectious disease detection system 160 can eliminate that infection likelihood at the step 270.

In an alternate embodiment, the infectious disease detection system 160 can count the individual 110 as infected, if the infection threshold is satisfied by one of the infection likelihoods (or an infection likelihood weighted by the corresponding confidence level). The infection threshold corresponds to a minimum infection likelihood that would trigger a definitive determination of the infection. Furthermore, the infectious disease detection system 160 can determine whether the infection likelihood (or an infection likelihood weighted by the corresponding confidence level) satisfies a first infection threshold. When the infectious disease detection system 160 determines that at least one infection threshold is satisfied, the infectious disease detection system 160 can consider the condition as high infection or severe infection.

The process 200 terminates at step 280.

In some other embodiments, the infectious disease detection system 160 can determine different levels of infection by accessing data from the clinical analyzer 134, the hematology analyzer 136, and the X-ray 126. The clinical data, CT scan, and X-ray provide additional parameters for further investigation of the infectious diseases specifically COVID-19.

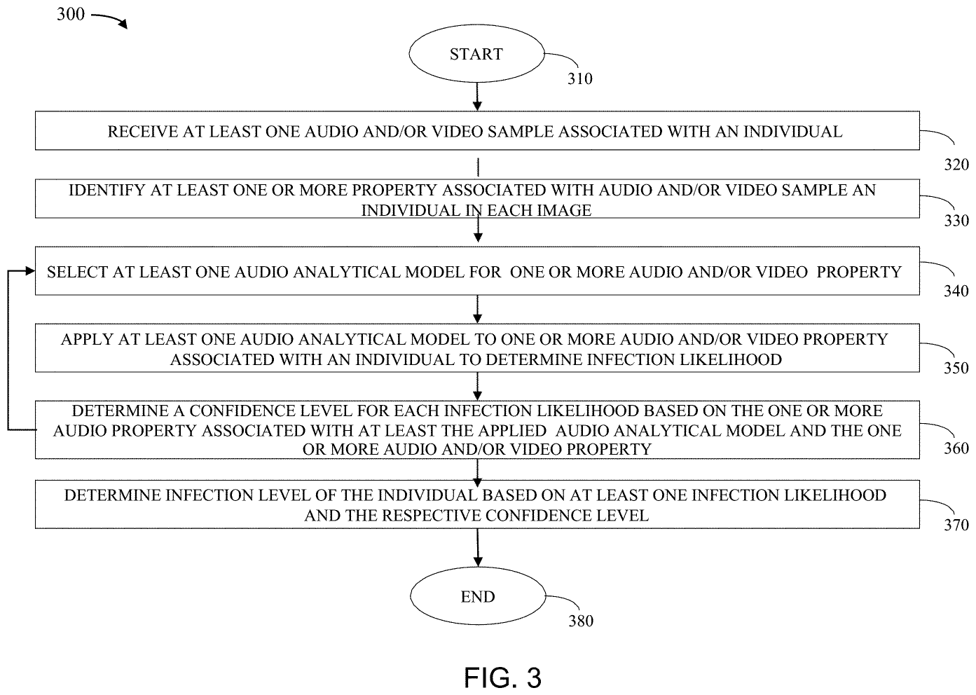

FIG. 3 illustrates the process 300 for determining infection of the individual 110 using audio and/or video data in an embodiment of the present invention. The process 300 is initiated at step 310 and immediately moves to step 320.

At step 320, the process 300 receives at least one audio and/or video sample or audio recording associated with the individual 110 for detection of the infectious disease. The audio and/or video sample or audio recording can include audio and/or video sample data one or more individuals 110.

In some embodiments, when the process 300 determines that multiple individuals are involved in the audio and/or video sample, audio and/or video sample can apply voice recognition to the audio recording to identify the segments or frames associated with a particular individual, for example individual X. The segments where the particular individual X is of interest and is the predominant speaker are marked. Similarly, when the process 300 determines that multiple individuals are involved in the video sample, the process 300 can apply image recognition to the video recording to identify the segments or frames in which the individual 110 is of prime interest. In some embodiments, both the audio segment and the video segment are of the particular individual of interest may be identified and processed in parallel or separately.

At step 330 of the process 300, the infectious disease detection system 160 identifies at least one audio property of the at least one audio recording of the particular individual X for further analysis. In addition, the process 300 analyzes at least one video property from the video recording. The analysis of video sample follows the process 200 with some or no modifications as described.

In embodiments, different properties of audio data and/or image frames can be analyzed for determination of infection in the individual 110. Example properties for audio and/or video can include, but are not limited to, loudness, jitteriness, and/or pitch, pixel density, pixel intensity, pixel color, image quality and other parameters. In some embodiments the infectious disease detection system 160 can identify various spectral features, such as spectral centroid to represent a center of mass of a spectrum (e.g., based on various different methods such as linear, logarithmic or exponential power of the spectrum); spectral flatness (typically measured in decibels) to represent how similar the audio is to noise, as opposed to tone, spectral complexity to represent a number of peaks in the spectrum; spectral contrast to represent aspects of the spectral peak, spectral valley, and the difference between the spectral peak and valley in each frequency sub-band; spectral roll-off to represent an amount of the right-skewedness of the spectrum; and spectral flux to represent how quickly a power spectrum of an audio signal is changing. The spectral flux can, in some embodiments, be determined by comparing the power spectrum for one frame against the power spectrum from a previous frame.

In some embodiments, the infectious disease detection system 160 may implement algorithms such as but not limited to mel-frequency cepstral coefficients (MFCCs), Bark-frequency cepstral coefficients (BFCCs), and/or gammatone frequency cepstral coefficients (GFCCs) from a type of cepstral representation of the audio clip. Mel-frequency cepstral coefficients can involve filters with center frequencies that are spaced along the mel scale, bark-frequency cepstral coefficients can involve filters with center frequencies spaced along the bark scale, and gammatone frequency cepstral coefficients can involve filters with center frequencies along the gammatone scale.

In some embodiments, the infectious disease detection system 160 can determine linear predictive coefficients (LPCs) and associated reflection coefficients of a signal; energies or magnitudes in equivalent rectangular bandwidth (ERB) bands of a spectrum using an equivalent rectangular bandwidth scale, for example; a sensory dissonance of an audio signal based on a roughness of the spectral peaks of the audio samples to represent a perceptual roughness of the sound; a ratio between the odd and even harmonic energy of a signal, with respect to the harmonic peaks of the signal; and tristimulus values of the audio sample with respect to its harmonic peaks to represent a mixture of the harmonics in a sound. Different variations of the tristimulus values can be used, such as a tristimulus value that represents a relative weight of the first harmonic, a tristimulus value that represents a relative weight of the second, third, and fourth harmonics; and a tristimulus value that represents the relative weight of all the remaining harmonics.

In some embodiments, the infectious disease detection system 160 can determine a mean and standard deviation of the fundamental frequency (F0Hz); a Harmonics-to-Noise (HNR) ratio to represent a measure of the proportion of harmonic sound to noise in the voice measured in decibels; mean and median of formants, which correspond to a concentration of acoustic energy around a particular frequency in the speech wave; average absolute difference between consecutive periods, divided by the average period; average absolute difference between consecutive periods (which can be represented in seconds); a relative average perturbation to represent an average absolute difference between a period and the average of it and its two neighbors, divided by the average period; a five-point period perturbation quotient to represent an average absolute difference between a period and the average of it and its four closest neighbors, divided by the average period; an average absolute difference between consecutive differences between consecutive periods, divided by the average period; an average absolute difference between the amplitudes of consecutive periods, divided by the average amplitude; an average absolute base-10 logarithm of the difference between the amplitudes of consecutive periods, multiplied by 20; a three-point amplitude perturbation quotient to represent the average absolute difference between the amplitude of a period and the average of the amplitudes of its neighbors, divided by the average amplitude; a five-point amplitude perturbation quotient to represent the average absolute difference between the amplitude of a period and the average of the amplitudes of it and its four closest neighbors, divided by the average amplitude; a 1-point amplitude perturbation quotient to represent an average absolute difference between the amplitude of a period and the average of the amplitudes of it and its ten closest neighbors, divided by the average amplitude; and an average absolute difference between consecutive differences between the amplitudes of consecutive periods.

The infectious disease detection system 160 can identify a property that relates to the entire audio recording, such as the fundamental frequency for the entire audio recording, or a property related to a segment or frame of the audio recording. For example, the audio property may be an average loudness for a particular segment of the audio recording in which the individual 110 is speaking.

In some embodiments, the infectious disease detection system 160 can preprocess the audio recording. For example, the infectious disease detection system 160 may remove portions of the audio recording associated with no or minimal speech. In some embodiments, the infectious disease detection system 160 may adjust the voice recording to enhance the audio properties, such as the equalization, volume, sampling rate, balance, and/or tone.

At step 340 of the process 300, the infectious disease detection system 160 selects at least one audio analytical model for using the audio property to detect the presence of the infectious disease in the individual 110. In another embodiment, the infectious disease detection system 160 selects at least one audio analytical model for using the audio property to detect the presence of the infectious disease in the individual 110 and further select at least one image analytical model to use at least one feature associated with the video sample of the individual 110 to detect the presence of infectious disease. The analysis of the video frame comprising an image can be performed as detailed in process 200. The process 200 can be followed herein in parallel.

The audio analytical models are stored in the analytical models 170. The audio analytical model may relate features or one or more audio properties identified in the audio recording to detect the infection level in the individual 110. In embodiments, different audio analytical models can be developed for different audio properties. For example, an audio analytical model can be developed for the audio property, that is, loudness. The high loudness in the audio recording may relate to continuous coughing, which may indicate that the individual 110 has high infection level. In another example, a different audio analytical model can be developed for those with no infection. In some embodiments, the infectious disease detection system 160 can develop audio analytical models for each property when there is no infection. During prediction of the infectious disease in the individual 110 using an audio analytical model, an audio recording that does not fit well within the outcome of prediction that the individual has no infection can be classified as infected. The level of infection may be determined using other audio properties in order to categories the individual 110 under different level of infection levels such as low infection, moderate infection and high infection.

In some embodiments, the infectious disease detection system 160 can generate the audio analytical model based on a set of training audio recordings. The training audio recordings can be stored in the data storage 166 or the analytical model database 168. The set of training audio recordings can include one or more audio recordings associated with different individuals, and each audio recording can be associated with the infection level. The infectious disease detection system 160 can then generate the audio analytical model based on the training audio recordings and the infection level associated with each training audio recording. For example, the infectious disease detection system 160 can generate the audio analytical model by applying a pattern recognition algorithm to the set of training audio recordings to detect the infection level. The one or more infection levels associated with one or audio recording properties establish patterns and/or relationship between the features identified in the training audio recordings and the associated infection levels. In some embodiments, multiple pattern recognition algorithms can be applied.

Example pattern recognition algorithms can include, but are not limited to, techniques based on nearest neighbor, k-nearest neighbors, support vector machines, naive Bayesian, decision trees, random forests, logistic regression, gradient boosting algorithms (e.g., XGBoost), and/or linear discriminant analysis.

At step 350 of the process 300, the infectious disease detection system 160 applies the at least one audio analytical model to the audio recording and/or at least one feature to the video sample and/or per frame of the video sample to determine an infection likelihood in the individual 110. The audio analytical model can generate infection likelihood for the audio property based on how well certain properties of the audio recording fit with the audio analytical model. Likewise, the analytical model can generate infection likelihood for each feature of the image based on how well certain image features or properties of the video recording fit with the analytical model.

At step 360 of the process 300, the infectious disease detection system 160 determines the confidence level for each of the infection likelihood based on characteristics associated with at least the audio analytical model and the audio property. In some embodiments, the analytical model for image analysis based on at least the image features or properties of the video recording may be performed in parallel. The process for image analysis is detailed in FIG. 2 and the same process is followed here.

In some embodiments, the audio data and the image data may be performed in parallel by selecting either same or different analytical models and can be combined using another analytical model for detecting the presence of the infectious disease in the individual 110.

In some embodiments, the infectious disease detection system 160 can determine, which aspects of the audio recording and the analytical model can affect the reliability of the resulting infection likelihood and the infectious disease detection system 160 can accordingly adjust the confidence level.

In embodiments, the steps 340, 350, and 360 can be iteratively performed by the infectious disease detection system 160 for each audio property of the audio recording identified at 330.

At step 370, the infectious disease detection system 160 defines the infection of the individual based on infection likelihood and the associated confidence level. Similar to step 270 of process 200, the infectious disease detection system 160 can define the infection of the individual 110 based on various methods and factors as described. Based on the determination, the infectious disease detection system 160 can generate the infection indicator to represent the infection level accordingly.

The process 300 terminates at step 380 after the infection level of the individual has been ascertained with a certain confidence level.

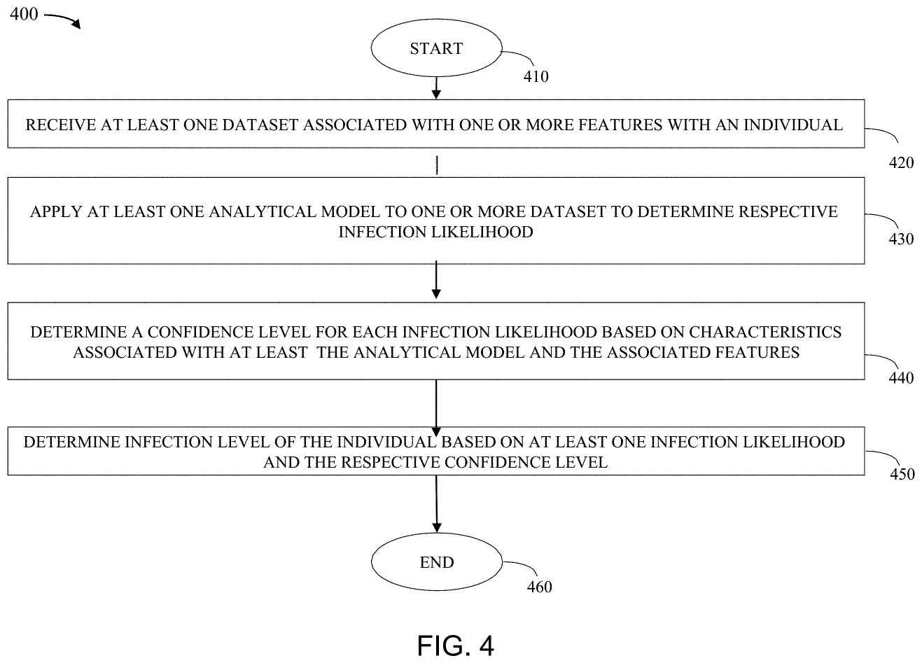

FIG. 4 illustrates a flow chart illustrating an exemplary process 400 for operating the infectious disease detection system 160 to detect the infection in the individual 110.

The process 400 is initiated at step 410. At step 420, the infectious disease detection system 160 receives at least one dataset associated with one or more features related to the individual 110. In various embodiments, the dataset can include data associated with more than one feature related to the individual 110. For example, the dataset can include physiological and/or vital sign measurements of the individual 110, such as, but not limited to, a heart rate recording, hydration levels, and/or an electrocardiogram (ECG) recording, CT scan, x-ray, clinical test reports and other type of medical test reports. The dataset can also include other data, such as images, videos, and/or audio recordings involving the individual 110. The infectious disease detection system 160 can consider multiple types of data when detecting the infection in the individual 110. For example, the infectious disease detection system 160 can detect infection level based on one or more input devices 120 that provide one or more parameters. Each parameter has one or more features associated with it. For example, the infectious disease detection system 160 can detect the infection level based on thermal image of various parts of the body such as hands, face, and eyes. In another example, the infectious disease detection system 160 can detect the infection level based on CT-scan. In another exemplary embodiment, the infectious disease detection system 160 can detect the infection level based on CT-scan. In another exemplary embodiment, the infectious disease detection system 160 can detect the infection level based on clinical data. In another exemplary embodiment, the infectious disease detection system 160 can detect the infection level based on an X-ray. In another exemplary embodiment, the infectious disease detection system 160 can detect the infection level based on hematology analysis.

In other exemplary embodiments, the infectious disease detection system 160 can detect the infection level based on one input device 120 or a combination of different input device(s) 120 or all the input device(s) as may be required depending upon the load factor of the infectious disease detection system 160.

At step 430, the process 400 applies at one analytical model to one or more datasets associated with the individual to determine the likelihood of infection. The process 400 may utilizes at least one analytical model from the analytical models 170 and apply the selected analytical model to the received processed data to predict the likelihood of the infection.

At step 440, the process 400 may associate a confidence level for infection likelihood based on the characteristics associated with the selected analytical model and the associated features. In some embodiments, the selected analytical model may estimate the likelihood of infection for each feature for prediction of the infectious disease associated with the individual. This process may be performed by the selected analytical model for one or more features associated with one or more parameters for each of the input devices 120.

At step 450, the process 400 may determine the infection level based on at least the infection likelihood along with the associated confidence level to arrive at final result for detection of the infections disease associated with the individual 110. In some embodiments, the infection level estimated for each feature based infection likelihood associated confidence level may be evaluated for one or more parameters associated with one or more input devices 120 to arrive at final result.

The process 400 terminates at step 460.

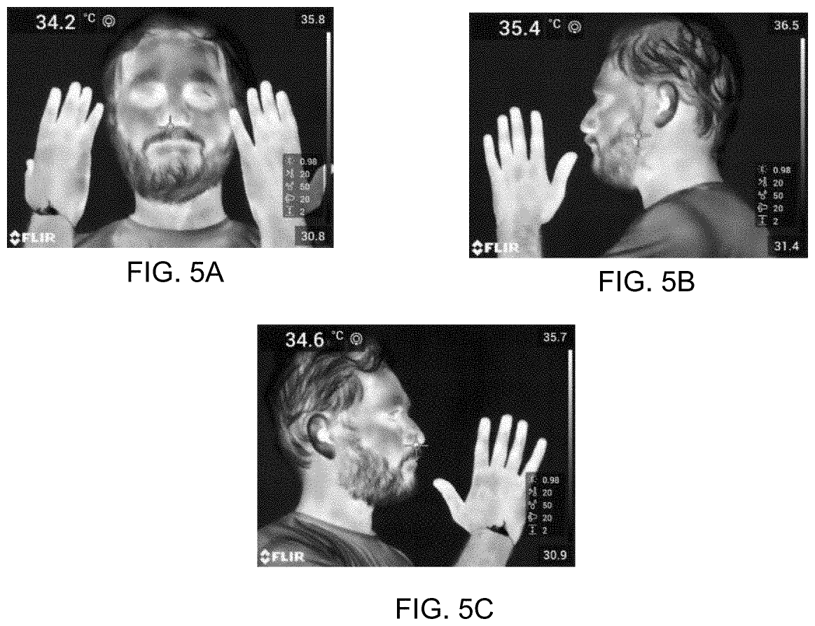

FIG. 5A, FIG. 5B and FIG. 5C illustrate different images of an individual as parameters using a thermal camera for detecting the infection level. FIG. 5A provides the front view of the face and the hands of the individual 110 captured by a thermal camera 122. The captured image is provided to the infectious disease detection system 160 for detection of the infectious disease. Likewise, the left side of the captured image is shown in the FIG. 5B and the right side view is provided in the FIG. 5C. FIG. 5A, FIG. 5B and FIG. 5C provide one or more parameters to infectious disease detection system 160. Furthermore, the parameters provide one or more features for the analytical models to detect the presence of the infectious diseases in the individual 110. Different views increase efficiency of the infectious disease detection system 160 by associating the likelihood of infection associated with one or more features with higher confidence level of assessment of the infectious disease.

In another embodiment, FIG. 5A, FIG. 5B and FIG. 5C provide one or more parameters for training and testing the infectious disease detection system 160. For example, the side view of right side and the left side provide higher confidence level of prediction of the infectious disease than by using only the front view. Different view from the thermal camera 122/RGB camera of the face and the hand may also be used to do the body temperature profiling of the individual 110.

In yet another embodiment, the images received from the RGB camera may be utilized for testing and/or training the infectious disease detection system 160.

In some embodiments, the image captured by the thermal camera/RGB camera is limited to exposed parts of the body such as neck, hands, and face. In embodiments, the data collected from different individuals may be utilized for data cleansing, for example, establishing benchmark and finding anomaly in the data such as outliners.

In some embodiments, the infectious disease detection system 160 uses the images of the hands, the neck and the front face, different section of the hands, different section of neck, different sections of face, and different orientation of face (left side view, right side view, and front view) to identify areas of interest and for calculating the profile in terms of contrast in temperature for detecting the infection level.

In some embodiments, the forehead, the eyelids, the nose, the lips, the cheek, the neck and other portions of the body of the individual 110 may be analyzed, compared with the hands, the fingers, finger and the nails of the individual 110 by the infectious disease detection system 160 to detect the level of the infection.

In some embodiments, the thermal image of supraorbital region around the eye (this is indicative of headache-it is the region right above the eye sockets) can be used by the infectious disease detection system 160 to detect the level of infection.

In some embodiments, the thermal image of the face can be used to identify fatigue or tiredness associated with an infectious disease such as COVID-19. For example, the rate of floe of blood, the level of activation of sweat glands, the metabolism of body cells, the blood pressure and other human parameters can be used by the infectious disease detection system 160 to detect the level of infection.

FIG. 6 illustrates the front and back view of the whole body scan of an individual to detect the infectious disease detection system using a thermal camera. The thermal image of FIG. 6 provides the temperature profile of the front side of the body and the backside of the body. In one embodiment, the data captured by the thermal camera 122 is utilized for training the infectious disease detection system 160 to detect the infection level in the individual 110.

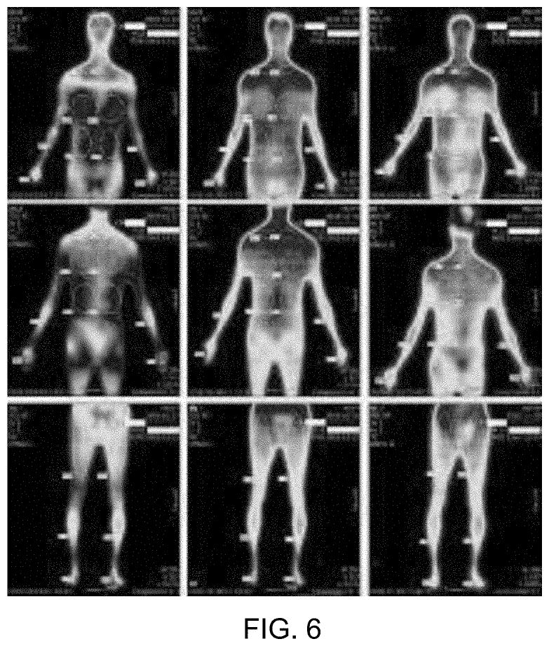

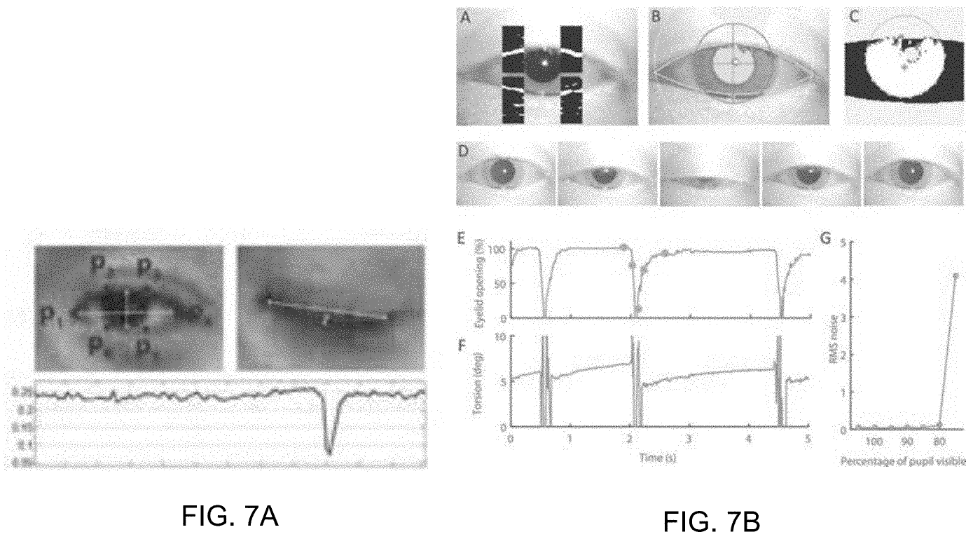

FIG. 7A and FIG. 7B illustrate the eye scan and/or the retina scan of an individual from an input device to the infectious disease detection system using a thermal camera and/or infrared camera or RGB camera for determining the redness in eye for detecting the infection level.

As shown in FIG. 7A and FIG. 7B, the eyes of the individual are used to identify the level of infection. For example, the infectious disease detection system 160 may identify eye from face image, identify pupil in the scanned image of eye for analyzing the patterns of whiteness and redness as shown in FIG. 7A. In one embodiment, the ratio of the white part of the eye (whiteness) may be compared with the red part of the eye (redness) to be used as one parameter with associated one or more features/characteristics to detect the level of infection.

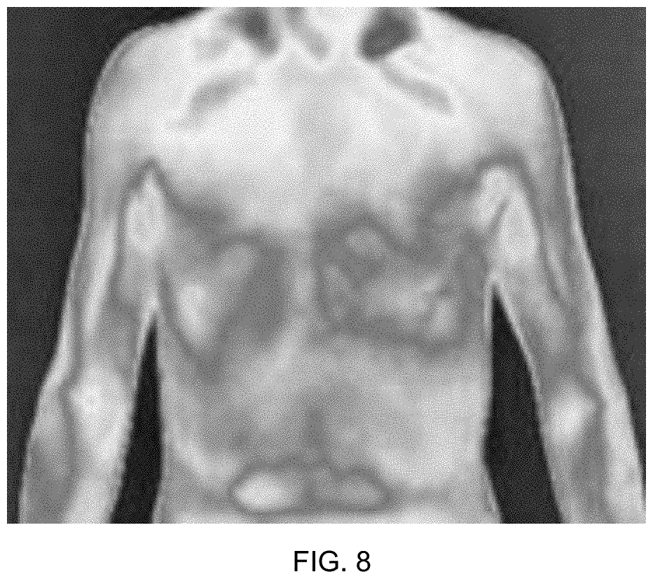

FIG. 8 illustrates the X-ray and/or CT-scan of chest of an individual provided as parameter to the infectious disease detection system 160 for detecting the infection level. The data from the X-ray or the CT-scan of the chest may be analyzed for areas of interest. The identified areas of interest in the X-ray image or a CT scan image may be passed to the selected analytical model and used for detection of the infectious disease.

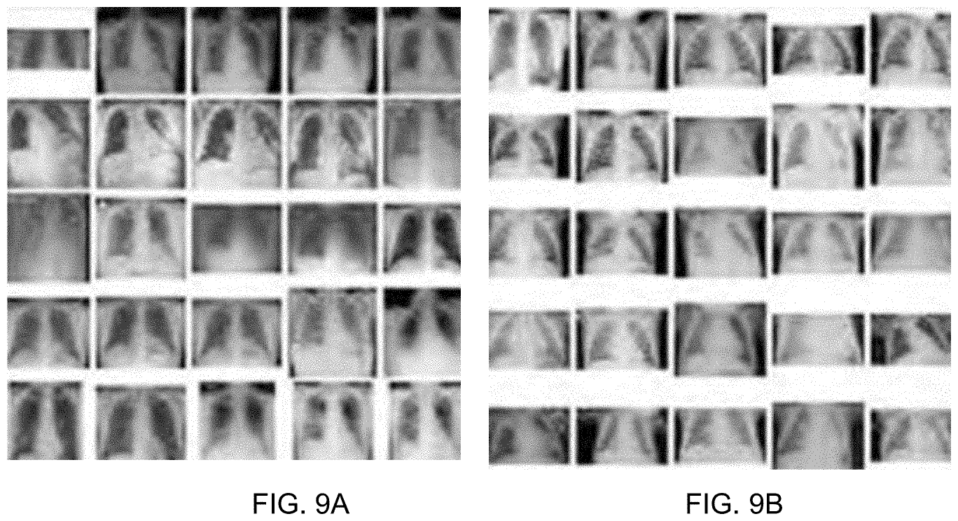

FIG. 9A and FIG. 9B illustrate the X-ray/CT Scan image of an individual provided as parameters for training the infectious disease detection system 160 for detecting the infection level of COVID-19 infection. FIG. 9A shows the X-ray/CT scan image of the individual with an infection and FIG. 9B shows the X-ray/CT scan image of the individual without any infection. The X-ray/CT scan image is provided to the infectious disease detection system 160 to detect the likelihood with reasonable accuracy if the individual is carrying the infection for COVID-19.

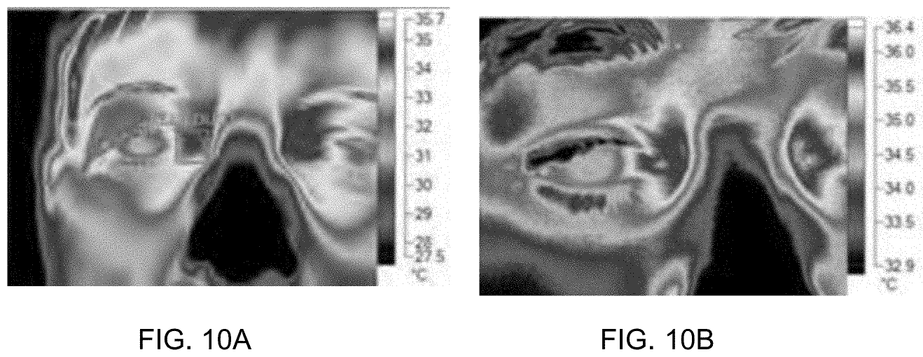

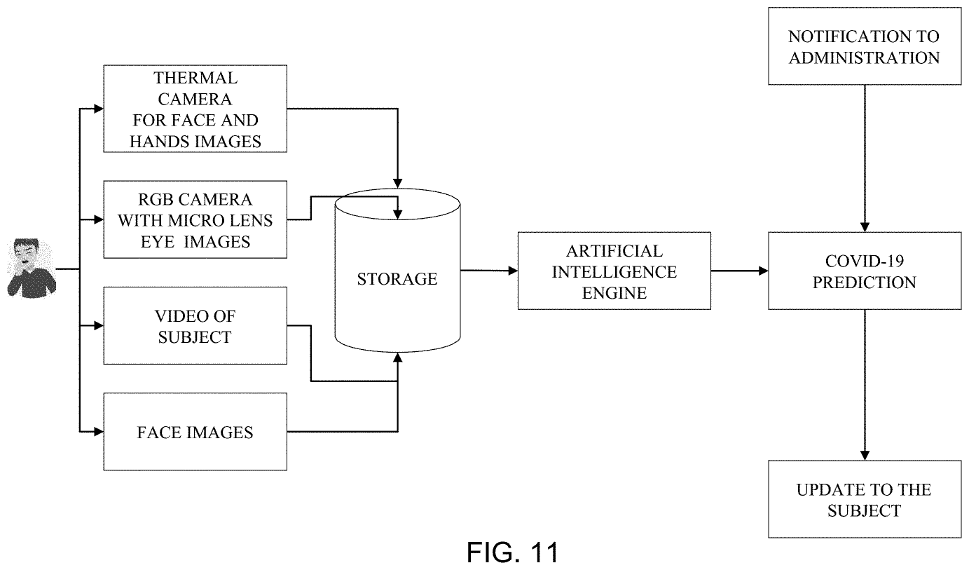

FIG. 10A and FIG. 10B illustrates a thermal image of the upper region of the face for detection of the infectious disease by the infectious disease detection system. FIG. 10A shows the thermal image of a healthy individual showing distribution of temperature on and around the nose of the individual with any infection. FIG. 10B shows the thermal image of an infected individual showing distribution of temperature on and around the nose of the individual inflected with flu and fever. The thermal image of the temperature distribution of the forehead and around the nose may be used to detect the infectious disease by the infectious disease detection system 160. Alternatively, the distribution of temperature on and around the nose of the individual can be used to train the infectious disease detection system 160 for predicting the infection in the individual.

In alternate embodiments, a RGB image of the upper region of the face, specifically the nose may be used for detection of the infectious disease by the infectious disease detection system 160 may be used as a parameter.

FIG. 11 illustrates the rapid screening architecture for detecting the infectious disease by the infectious disease detection system in another embodiment of the invention. In this exemplary embodiment, the infectious disease detection system 160 uses a screening process and a detection mechanism to predict whether an individual is infected by COVID-19 virus. The infectious disease detection system 160 comprises of thermal camera 122, and a RGB camera 132 and an audio/video recorder and analyzer 142 to determine the infection level of the individual. In addition, the thermal image captured by the thermal camera 122 and the RGB image captured by the RGB camera 132 can be analyzed to evaluate the distribution of the body temperature to indicate the severity of the disease. In embodiments, the image data of the individual in the form of thermal image and/or the RGB images of the face, hands and eyes can be used to detect the level of infection. In a different embodiment, the audio/video recorder and analyzer 142 can detect and highlight coughing and sneezing of the infected individual. In another embodiment, the audio/video recorder and analyzer 142, the thermal image and/or the RGB camera can be used to detect the level of infection in the individual 110 by the infectious disease detection system 160.

Initially, the infectious disease detection system 160 performs the data collection and building an artificial intelligence model for prediction of the individual for detection of the infectious disease as already described in various embodiments. In one embodiment, a data set for training the infectious disease detection system 160 is provided. The training dataset may be obtained from the diagnosis by the laboratory, hospital, medical centers and physicians or other sources involved with detection of infectious disease. Different features associated with one or more parameters may be analyzed for determining the likelihood of infection with a predefined confidence level. For example, the infectious disease detection system 160 to identify credentials of the individuals the associated medical history associated, the infectious disease detection system 160 may utilize face recognition identification algorithms to identify and locate the individual, who is currently being scanned by the infectious disease detection system 160.

In some embodiments, the subsequent phases will be focused on scaling the solution across the globe and making any necessary changes to the analytical model. After performing the fine-tuning, the infectious disease detection system 160 can be used for scanning, quarantine and medical treatment of the individuals that are infected with the infectious disease such as COVID-19 and are at high risk of spreading of the disease.

In embodiments, when the system has been trained, the infectious disease detection system 160 can be utilized for screening and detection of infected individuals and further for prediction of individual infected with the COVID-19 infection. The infectious disease detection system 160 with the artificial intelligence backed prediction model will be able to detect potential contamination in terms of virus in the individuals and initiate a rapid action of quarantine and/or medical procedures. The data collected in the first phase may be utilized as a training set further to develop the analytical model for better accuracy and response.

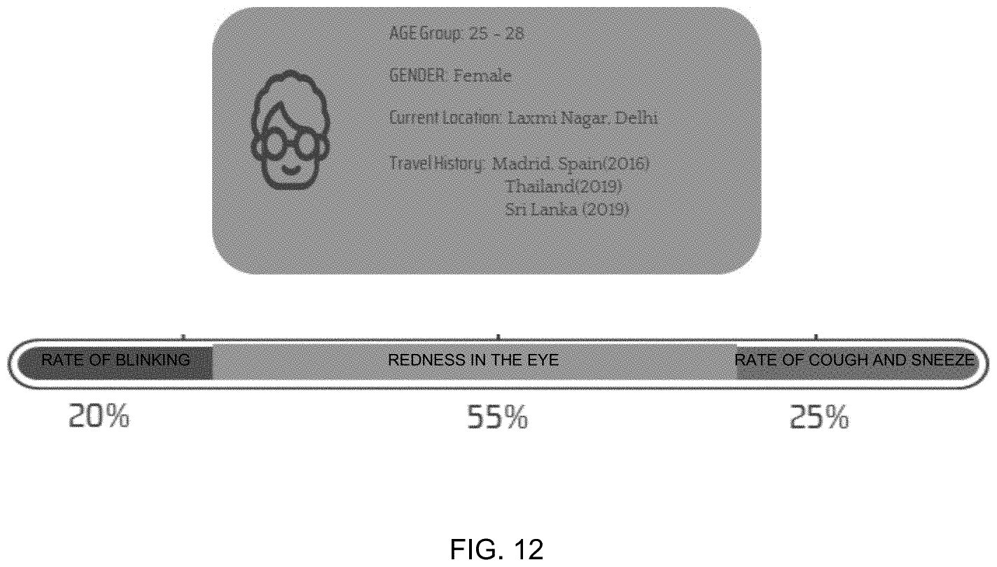

FIG. 12 illustrates a scan of face of the individual for prediction of an infectious disease in an embodiment of the present invention. The scanned image or the scanned video of face including the eyes are analyzed for one or more parameters and features such as rate of blinking of eye, redness in eye, and rate of cough. FIG. 12 shows an exemplary case of different percentage attributed to different parameters and/or one or more features/characteristics, wherein the rate of blinking of eye is given 20% weightage, redness in eye is given 55% weightage, and rate of cough is given 25% weightage. In other embodiments, the different percentage attributed to different parameters and/or one or more features/characteristics may be different.

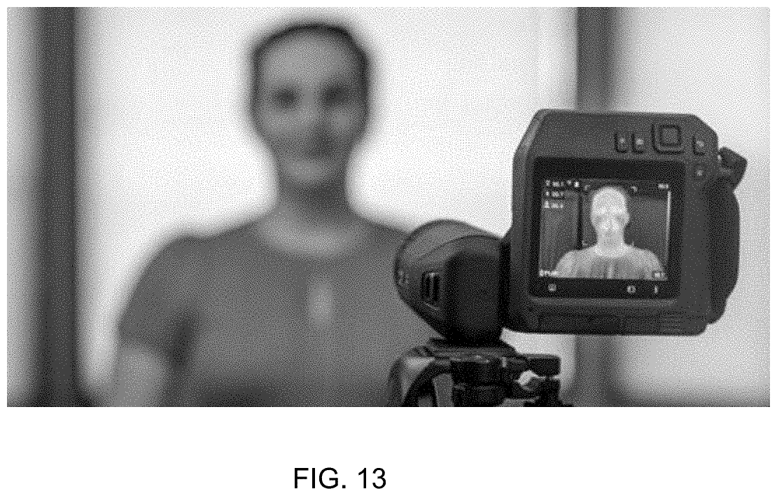

FIG. 13 illustrates the scan of upper body for detecting the infected individual using a thermal camera in another embodiment of the present invention. The thermal image of the upper chest can be used as an additional input in detecting the infectious disease along with thermal camera, RGB camera, and audio/video recorder & analyzer. In this embodiment at least one of the analytical models is trained to detect the infectious level by scanning the thermal distribution of the spots in the lungs and the chest. The distribution of infection may depend upon the size and density of the spots in the lungs, which may indicate high infection level.

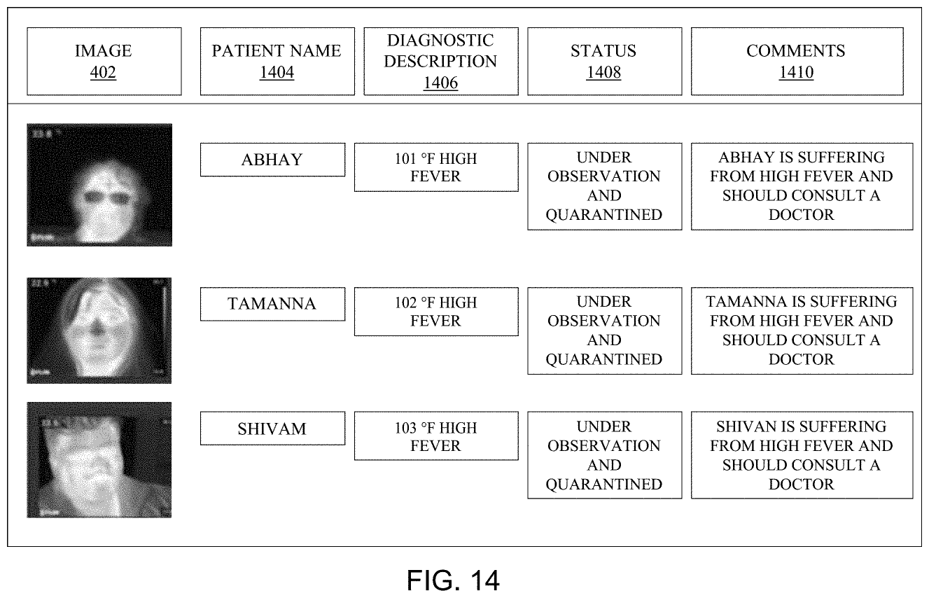

FIG. 14 illustrates a user interface of the infectious disease detection system 160 in an embodiment of the present invention. The user interface may include previous test histories of the individual. Additionally, the user interface may different attributes of the individual 110 such as image 1402, name 1404, diagnostic description 1406, status 1408, and comments 1410 related to history and the present condition of the individual. In addition, the user interface may also be connected with the clinical tests and disease related histories of the individual 110. In addition, the user interface allows tracking of the patients by GIS, for example, the travel history, current location, contact list, and places he visited in the past and other tracking requirement.

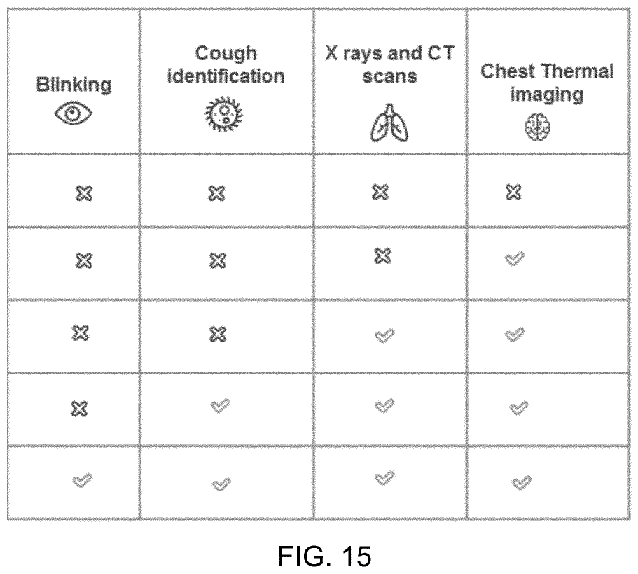

FIG. 15 illustrates the evaluation of some of the parameters by the infectious disease detection system 160 in an embodiment of the present invention. The evaluation parameters include blinking rate of eye, cough identification, X-ray and/or CT-scans and the thermal imaging of the chest. Each of these parameters provides prediction for presence of the infectious disease in the individual 110. Although only four parameters are illustrated in the exemplary embodiment, in other variations more than or less than four parameters may be evaluated of the infectious disease such as but not limited redness of eye, sneezing, fever, cold, thermal distribution of face including nose, eyes, lips and ears, clinical reports, thermal image of chest and lungs and other parameters for detection of the infectious disease,

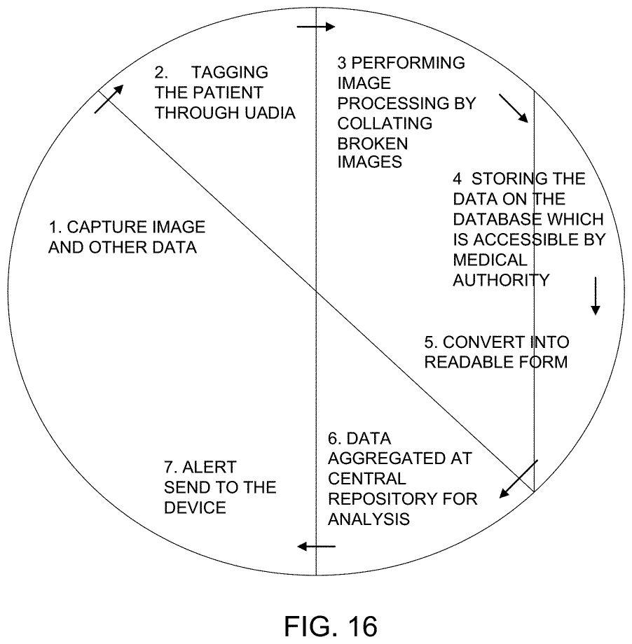

FIG. 16 illustrates the data flow of the process of scanning and detection of the infectious disease detection system for tracking and management in another embodiment of the invention.

The infectious disease detection system 160 may capture data from one or more input devices as a first step. As second step, the infectious disease detection system 160 may use face recognition algorithm implemented in the infectious disease detection system 160 to identify the individual for tagging it through unique identity or social security number. In some embodiment, the unique identification may be selected based on the system prevalent in that country. The unique identity associated with the individual may be utilized for tagging the individual. At step three, the infectious disease detection system 160 may perform image analysis and processing by collating the broken images to gather one or more parameters and the associated one or more feature to be applied to the infectious disease detection system 160. At step four, the gathered data of the tagged individual may be stored in the database, which may be accessible to the medical authorities. At this step, in some embodiments, the infectious disease detection system 160 one or more parameters and the associated one or more feature using the artificial intelligence model to predict the if the tagged individual is suffering from an infectious disease and then store the data along with the current state, that is either infected or non-infected. This may be utilized for contact tracing.

At step five, the infectious disease detection system 160 may convert the gathered data at step 3 in readable form. At step 6, the stored data may be passed to a cloud server for storing for analysis. In some embodiments, the prediction that the tagged individual is suffering from an infectious disease is performed at this step. Finally, at step seven, an alert that the tagged individual is suffering from an infectious disease specifically COVID19 is passed to the relevant stake holder for further action.

What is claimed is:

1. A computer implemented method for rapid scanning, surveillance and detection of an infectious disease in an individual, the computer implemented method configured to perform the steps of:
receiving at least one parameter associated with the individual as an input from at least one input device;
identifying at least one feature in the received parameter;
analyzing the received parameter and the identified feature using an artificial intelligence implemented algorithm previously trained to create one or more analytical models for detection of infectious disease;
selecting one analytical model from one or more analytical models based on a pre-determined criterion;
applying the selected analytical model for estimating a likelihood of the individual having the infection within a certain confidence level to classify the individual as infected with the infectious disease, and
providing a report of presence of the infectious disease.

2. A computer implemented method of claim 1, further determining one or more characteristics associated with the received parameter, wherein the determined characteristics may be utilized for detection of the infectious disease.

3. A computer implemented method of claim 1, wherein the one or more input devices comprise at least one of a thermal camera, a CT scanner, a X-ray machine, an infrared camera, an RGB camera, a clinical analyzer, a hematology analyzer, a temperature sensor and an audio video recorder and analyzer.

4. A computer implemented method of claim 1, wherein the received parameter associated with the input device is at least one of a temperature, a RGB image, a thermal image, an audio data, a video data, an audio/video data, or a clinical test report.

5. A computer implemented method of claim 1, wherein at least one received parameter is an RGB image of the face of the individual and different section of the RGB image represent one or more features.

6. A computer implemented method of claim 1, wherein the pre-defined criterion is determined from at least one of the received parameter, the identified feature, a characteristic of the received parameter, the individual's gender, the individual's age, and the individual's medical history.

7. A computer implemented method of claim 1, wherein at least one received parameter is an audio video recording of the individual and the audio video recording has one or more features, wherein at least one identified feature is associated with the audio recording and at least one other identified feature is related to the video recording.

8. A computer implemented method of claim 1, wherein at least one received parameter is a body temperature of the individual.

9. A computer implemented method of claim 1, wherein at least one received parameter is a CT scan image of the individual and wherein at least one identified feature is the CT scan of the lungs.

10. A computer implemented method of claim 1, with additional steps before the step of providing a report of presence of the infectious disease, such steps comprising:
applying at least a second selected analytical model for estimating the likelihood of the individual having the infection within a certain confidence level to classify the individual as infected with the infectious disease;
aggregating the classification of each application of selected analytical models to detect infection within a certain confidence level to ascertain the likelihood of infection; and providing a report for presence of the infectious disease in the individual.

11. An infectious disease detection system for rapid scanning, surveillance and detection of an infectious disease in an individual, the infectious disease detection system comprising:
one or more input devices;
a parameter analysis module associated with the one or more input devices to derive at least one parameter;
a feature analysis module associated with the parameter analysis module to derive at least one feature associated with the at least one derived parameter;
an analytical model database having at least one analytical model, wherein the analytical models have been previously used to detect the presence of an infectious disease;
an analytical model selector module associated with the analytical model database to select one analytical model based on a pre-determined criterion;
a detection module associated with a processor to apply the selected analytical model to detect the presence of the infectious disease and to produce a report.

12. The infectious disease detection system of claim 11, wherein the one or more input devices comprise at least one of a thermal camera, a CT scanner, a X-ray machine, an infrared camera, a clinical analyzer, a hematology analyzer, a temperature sensor and an audio-video recorder and analyzer.

13. The infectious disease detection system of claim 11, wherein at least one derived parameter associated with the one or more input devices is at least one of a temperature, a RGB image, a thermal image, an audio data, a video data, an audio/video data, or a clinical test report.

14. The infectious disease detection system of claim 11, wherein at least one derived parameter is an RGB image of different parts of the face.

15. The infectious disease detection system of claim 11, wherein at least one derived parameter is the audio video recording of the individual.

16. An infectious disease detection system of claim 11, wherein the detection module associated with the processor further comprises:
an aggregator module configured to detect the presence of infectious disease with each parameter and applying to the outcome of each parameter a different analytical model and aggregating the results to produce a report.

17. A computer readable medium for implemented method for rapid scanning and surveillance of an infectious disease in an individual having encoded instructions there upon, when executed by a processor is configured to perform the steps of:
receiving at least one parameter as an input from one or more input devices;
identifying at least one feature in the at least one parameter associated with the individual;
analyzing at least one parameter and the associated feature using artificial intelligence implemented algorithms previously trained to create one or more analytical models for detection of infectious disease;
selecting at least one analytical model based on a pre-determined criterion;
applying the selected analytical model for estimating the likelihood of the individual having the infection within a certain confidence level to classify the individual as infected with the infectious disease, and
providing a report of presence of the infectious disease.

18. A computer readable medium of claim 17, wherein the one or more input devices comprise at least one of a thermal camera, a CT scanner, a X-ray machine, an infrared camera, a clinical analyzer, a hematology analyzer, a temperature sensor and an audio-video recorder and analyzer.

19. A computer readable medium of claim 17, wherein at least one received parameter is one of a temperature, a RGB image, a thermal image, an audio data, a video data, an audio/video data, or a clinical test report.

20. A computer readable medium of claim 17, wherein at least one received parameter is an RGB image of different parts of the face.
